(19) United States
(12) Patent Application Publication
(10) Pub. No.: US 2026/0284171 A1
(43) Pub. Date: Sep. 24, 2026

(54) COMPOSITION

(71) Applicant: Oxford University Innovation Limited, Oxford (GB)

(72) Inventors: Christoph TANG, Oxford (GB); Rebekah JONES, Northampton (GB); Ana CEHOVIN, Oxford (GB)

(21) Appl. No.: 19/475,441

(22) PCT Filed: Apr. 19, 2024
(86) PCT No.: PCT/GB2024/051029

§ 371 Date: Oct. 15, 2025

(30) Foreign Application Priority Data
Apr. 20, 2023 (GB) 2305851.4

(51) Int. Cl. A61K 39/095; A61K 39/00; A61K 39/39; A61P 37/04; C07K 14/22

(52) U.S. Cl. CPC A61K 39/095; A61K 39/39; A61P 37/04; C07K 14/22; A61K 2039/55505; A61K 2039/575

(57) ABSTRACT

The invention relates to a composition comprising OMVs obtained from a modified Neisseria gonorrhoeae strain expressing a PorB variant, methods for preparing said composition, and its uses including vaccines.

### FIELD OF THE INVENTION

The invention relates to compositions, such as vaccines against Neisseria bacteria, and methods of preparing said compositions.

### BACKGROUND TO THE INVENTION

Neisseria gonorrhoeae causes the sexually transmitted infection gonorrhoea and has developed resistance to all known treatments. When left untreated, Neisseria gonorrhoeae infection can lead to severe complications including pelvic inflammatory disease, infertility, ectopic pregnancy and neonatal blindness. Additionally, there is clear evidence that gonorrhoea facilitates the acquisition and spread of HIV. Infection with Neisseria gonorrhoeae is highly prevalent in low- and middle-income countries. Therefore, the control of gonococcal infection would have a major impact on the sexual and reproductive health of individuals living in impoverished circumstances. Neisseria gonorrhoeae has a remarkable ability to develop resistance to antibiotics, through the acquisition of plasmids and chromosomal mutations.

Currently, there is no vaccine available against Neisseria gonorrhoeae, with whole cell and single protein subunit vaccines proving unsuccessful in the past. The development of vaccines against Neisseria gonorrhoeae is hampered by challenges. For example, natural infection of Neisseria gonorrhoeae does not provide immunity, as Neisseria gonorrhoeae suppresses innate and adaptive immune responses. Hence, there are no correlates of protection. Furthermore, Neisseria gonorrhoeae has an extraordinary capacity to vary the composition of its cell surface through phase variation and antigenic variation, thus making it difficult to identify antigens useful for vaccines. However, a key issue remains that some gonococcal antigens are capable of suppressing immune responses.

There has been a resurgence of interest in vaccines against Neisseria gonorrhoeae as retrospective analysis revealed evidence of cross-protection against gonorrhoea after immunisation with a Neisseria meningitidis outer membrane vesicle (OMV) vaccine. However, the effectiveness of this meningococcal vaccine against Neisseria gonorrhoeae was estimated to be low, at ~31%. It was considered cross-protection against Neisseria gonorrhoeae elicited by meningococcal OMVs may be attributed to the similarity of cell surface proteins between these species.

Therefore, it is an object of the invention to develop a further or improved immunogenic composition, such as a vaccine, against Neisseria bacteria, in particular Neisseria gonorrhoeae.

### SUMMARY OF THE INVENTION

The inventors found that Neisseria gonorrhoeae outer membrane vesicles (OMVs) containing a variant PorB, such as PorB from a Neisseria species other than Neisseria gonorrhoeae, is capable of being an effective vaccine against Neisseria gonorrhoeae. In particular, the inventors created a modified Neisseria gonorrhoeae strain in which the endogenous Neisseria gonorrhoeae porB gene has been replaced with a Neisseria meningitidis porB gene, and the endogenous rmpM gene has been deleted. Surprisingly, OMVs obtained from the modified Neisseria gonorrhoeae strain elicited significantly higher antibody titres against model gonococcal antigens, as well as meningococcal antigens, when compared to OMVs obtained from a Neisseria gonorrhoeae strain that contains endogenous porB. OMVs obtained from the modified Neisseria gonorrhoeae strain are therefore useful as a composition for inducing an immune response against Neisseria bacteria. Such OMVs would therefore be useful as immunogenic compositions, such as vaccines, against Neisseria bacteria, such as Neisseria gonorrhoeae and Neisseria meningitidis, in particular Neisseria gonorrhoeae.

The inventors also found that OMVs obtained from a modified Neisseria gonorrhoeae strain of the invention were capable of eliciting a Th1-bias against a range of gonococcal antigens, compared to OMVs obtained from a Neisseria gonorrhoeae strain that contains endogenous porB. The inventors also found that OMVs obtained from a modified Neisseria gonorrhoeae strain of the invention were capable of eliciting a greater and more diverse total IgG response compared to OMVs obtained from a Neisseria gonorrhoeae strain that contains endogenous porB. Replacing gonococcal PorB with meningococcal PorB in N. gonorrhoeae derived OMVs was therefore shown to elicit enhanced humoral and cellular immune responses, including a Th1-skew. This further demonstrates that such OMVs would be useful as immunogenic compositions, such as vaccines, against Neisseria bacteria, such as Neisseria gonorrhoeae or Neisseria meningitidis, in particular Neisseria gonorrhoeae. The most abundant outer membrane protein in Neisseria gonorrhoeae is porin PorB, which facilitates ion exchange across the outer membrane. PorB represents a large proportion (~60%) of both outer membrane and

OMV proteomes. Neisseria gonorrhoeae Neisseria gonorrhoeae PorB was not previously considered to be a useful vaccine antigen because it is an extremely variable antigen, as well as potentially immunosuppressive. However, PorB has several immunomodulatory properties: i) resistance to killing by the complement system by recruiting host negative regulatory proteins, ii) influencing the innate immune response by inducing apoptosis in macrophages, and iii) influencing the adaptive immune response by inhibiting T cell proliferation.

PorB is essential for the viability of Neisseria gonorrhoeae. Therefore, rather than deleting the porB gene from Neisseria gonorrhoeae, the inventors replaced the Neisseria gonorrhoeae porB gene with a porB variant, e.g. porB from Neisseria meningitidis. Without wishing to be bound by theory, the potent immune response elicited by the OMVs obtained from the modified Neisseria gonorrhoeae strain may be attributed to the evasion of immune suppression that is commonly associated with Neisseria gonorrhoeae antigenic preparations containing Neisseria gonorrhoeae PorB. Furthermore, Neisseria meningitidis PorB displayed broad immunostimulatory properties when used as an adjuvant, able to induce antigen specific B cell and T cell responses. Thus, including such a PorB variant in the Neisseria gonorrhoeae OMVs would augment the immune response.

In addition, the potent immune response elicited by the OMVs obtained from the modified Neisseria gonorrhoeae strain may also be attributed to the absence of reduction modifiable protein (RmpM). RmpM forms a complex with PorB in the bacterial outer membrane. RmpM has been shown to result in blocking antibodies that prevent binding of bactericidal antibodies to other surface proteins, such as PorB. Furthermore, modified Neisseria gonorrhoeae strains which do not express RmpM generated larger amounts of OMVs, which is advantageous for producing large quantities of OMVs for use as immunogenic compositions, such as vaccines.

Accordingly, the invention provides an outer membrane vesicle (OMV) obtained or obtainable from a modified Neisseria gonorrhoeae strain expressing a PorB variant.

The invention also provides a Neisseria gonorrhoeae OMV comprising a PorB variant.

The invention also provides a modified Neisseria gonorrhoeae strain expressing a PorB variant.

The invention also provides a method of preparing a modified Neisseria gonorrhoeae strain disclosed herein.

The invention also provides a method of preparing an OMV disclosed herein.

The invention also provides an immunogenic composition comprising an OMV disclosed herein.

The invention also provides an immunogenic composition comprising a modified Neisseria gonorrhoeae strain disclosed herein, or fragments thereof.

The invention also provides an OMV disclosed herein, or an immunogenic composition disclosed herein, for use as a medicament.

The invention also provides a vaccine derived from a modified Neisseria gonorrhoeae strain described herein.

The invention also provides a method of treating a Neisseria infection in a subject by administering to the subject an effective amount of the OMV disclosed herein, or the immunogenic composition disclosed herein.

The invention also provides an OMV disclosed herein, or an immunogenic composition disclosed herein, for use in a method of preventing or treating a Neisseria infection in a subject, wherein the method comprises administering said OMV or immunogenic composition to the subject.

The invention also provides the use of the OMV disclosed herein, the modified Neisseria gonorrhoeae strain disclosed herein or the immunogenic composition disclosed herein, as a vaccine, a detecting agent or a reagent for raising antibodies.

### BRIEF DESCRIPTION OF THE FIGURES

FIG. 1 shows the generation of Neisseria gonorrhoeae (Ng) FA1090 expressing Neisseria meningitidis (Nm) MC58 porB. A) Schematic representation of replacing the Ng porB gene with Nm porB, under the control of the native Ng promoter. Kanamycin resistance cassette (kanR) under its own promoter is present downstream for selection. Dotted lines represent regions for homologous recombination. B) Western blot analysis confirming complete replacement of Ng PorB with Nm PorB, using specific monoclonal antibodies indicated. C) Genetic deletion of rmpM via insertion of an erythromycin cassette (eryR). D) Western blot confirming deletion of rmpM in both PorB backgrounds. E) Growth curve of mutant strains.

FIG. 2 shows: A) Representative image of a Coomassie-stained 12% SDS-PAGE separation of outer membrane vesicles (OMVs) obtained from Neisseria gonorrhoeae FA1090ΔrmpM (left lane) and FA1090_{MC58 PorB} ΔrmpM (right lane). Ng PorB is indicated ~35 kDa and Nm PorB at 37 kDa. B-C) Mouse immunisation groups and immunisation schedule used to ascertain the impact of Ng- or Nm-PorB expressing OMVs on the immune response to model antigen factor H binding protein (fHbp).

FIG. 3 shows IgG responses after immunisation with Ng- or Nm-PorB expressing OMVs. A-C) Endpoint IgG antibody titres of pooled sera recognising the model antigen fHbp, and the OMV antigens MtrE and MetQ, determined by ELISA. D-E) Western blot comparison of the expression of MtrE and MetQ in Ng- or Nm-PorB expressing OMVs.

FIG. 4 shows how the proteomes of OMVs derived from N. gonorrhoeae FA1090ΔrmpM and FA1090_{MC58 PorB}ΔrmpM differ. Proteins are listed in the figure according to the abundance ratio (Nm PorB OMVs/Ng PorB OMVs), with the lowest abundance ratio at the top, and the highest abundance ratio at the bottom. Proteins listed from “MtrE” downwards have an abundance ratio of ≥2. Proteins listed from “Pils” upwards have an abundance ratio of ≤0.5. Proteins listed from “NEIS1824” to “Cah” have an abundance ratio from 0.5 to 2.

FIG. 5 shows IgG responses after immunisation with Ng-or Nm-PorB expressing OMVs. A-C) Endpoint IgG1 and IgG2a titres recognising the model antigen fHbp, and the OMV antigens MtrE and MetQ, determined by ELISA (two bars on left hand side of graphs correspond to fHbp+Ng PorB OMVs, two bars on right hand side of graphs correspond to fHbp+Nm PorB OMVs). Data are the mean±standard deviation. *** p<0.001, *** p<0.0001.

FIG. 6 shows serum bactericidal activity against WT N. gonorrhoeae FA1090 after immunisation with Ng-or Nm-PorB OMVs. NHS: normal human serum (IgG and IgM depleted), HI: heat inactivated.

FIG. 7 shows antigen-specific total IgG responses in sera from individual mice immunised with Ng PorB OMVs (third column) or Nm PorB OMVs (fourth column), compared to PBS alone (first column) and fHbp alone (second column); each column is the result for an individual mouse. Individual antigens are in indicated in each row. Shading represents the mean fluorescence intensity (MFI) value for antibody binding to each antigen.

FIG. 8 shows antigen-specific IgG1 and IgG2a responses in sera from mice immunised with Ng PorB OMVs or Nm PorB OMVs. Individual antigens are in indicated in each row; each column is the result for an individual mouse. Each shading block represents the mean fluorescence intensity (MFI) value for antibody binding to each antigen.

FIG. 9 shows PCA analysis applied to antigen-specific total IgG responses. A) Separation by individual serum sample, including all antigens. The biplot is generated using the squared coordinates (cos²) for PC1 and PC2, calculated as the squared coordinates of the eigenvalues. B) Contributions of individual antigens, excluding PorB and Opa variants, to PC1 and PC2 separation.

FIG. 10 shows cytokines produced by murine splenocytes after immunisation with Ng-or Nm-PorB OMVs, when re-stimulated with concanavalin A (ConA, positive control), Ng PorB OMVs, Nm PorB OMVs or no stimulation. A) interferon-γ (IFNγ), B-F) interleukin (IL)-4, IL-10, IL-17A, IL-6 and IL-2. Data are the mean±standard error of the mean. * p<0.05, *** p<0.001, **** p<0.0001, ns: not significant. In each graph, for each re-stimulation condition, the data are ordered according to treatment groups, with immunisation with PBS alone, fHbp alone, fHbp+Ng PorB OMVs, and fHbp+Nm PorB OMVs, respectively from left to right.

FIG. 11 shows Coomassie-stained 12% SDS-PAGE separation of three independent replicates of outer membrane vesicles (OMVs) obtained from N. gonorrhoeae FA1090 strains as indicated ΔrmpM and FA1090_{MC58 PorB} ΔrmpM. Ng PorB is indicated ~35 kDa and Nm PorB at 37 kDa.

### BRIEF DESCRIPTION OF THE SEQUENCE LISTING

SEQ ID NO: 1 is the nucleotide sequence of Neisseria gonorrhoeae FA1090 porB.

SEQ ID NO: 2 is the nucleotide sequence of Neisseria meningitidis MC58 porB.

SEQ ID NO: 3 is the nucleotide sequence of Neisseria gonorrhoeae FA1090 rpmM.

SEQ ID NO: 4 is the amino acid sequence of Neisseria gonorrhoeae FA1090 PorB.

SEQ ID NO: 5 is the amino acid sequence of Neisseria meningitidis MC58 PorB.

SEQ ID NO: 6 is the amino acid sequence of Neisseria gonorrhoeae FA1090 RmpM.

SEQ ID NO: 7 is the nucleotide sequence of the Kanamycin resistance cassette (kanR).

SEQ ID NO: 8 is the nucleotide sequence of the Erythromycin resistance cassette (eryR).

SEQ ID NOs: 9-16 are predicted extracellular loops of Neisseria gonorrhoeae FA1090 PorB.

SEQ ID NOs: 17-24 are predicted extracellular loops of Neisseria meningitidis MC58

### PorB.

SEQ ID NOs: 25-38 are the nucleotide sequences of the primers used in the Examples.

SEQ ID NOs: 39-40 are the PorB peptide standards used in the absolute quantification of PorB in OMVs by mass spectrometry.

### DETAILED DESCRIPTION OF THE INVENTION

### Outer Membrane Vesicle (OMV)

The invention relates to OMVs formed from the outer membrane of modified Neisseria gonorrhoeae strains as described herein. OMVs are vesicles that are formed from the outer membrane of Gram negative bacteria and contain many components found within the external surface of the parent bacterium (e.g. see review by Lieberman, Front. Microbiol 2022, sec. Microbiol Immunology vol 13).

The OMV of the invention may be a native OMV, physically-extracted, or detergent extracted.

The OMV of the invention may be a native OMV. Hence, the OMV is released spontaneously from the Neisseria gonorrhoeae strain, e.g. during growth or environmental stress in vitro or in vivo. Such an OMV would be present in the culture medium and can readily be separated from the bacteria, as explained further below.

The OMV of the invention may be derived from mechanical disruption of the bacteria. Hence, the OMV of the invention may be a physically-extracted OMV. For example, the OMV may be released from the bacteria as a result of physical (non-detergent) disruption, such as extraction with EDTA, sonication, or vortexing.

The OMV of the invention may be derived from detergent extraction. Hence, the OMV of the invention may be a detergent-extracted OMV. The detergent may be deoxycholate and/or sodium dodecyl sulfate.

The OMV of the invention comprises bacterial antigens on its surface, for example 2, 3, 4, 5, 6, 7, 8, 9, 10 or more, 15 or more, 20 or more, 30 or more, 40 or more, or 50 or more different bacterial antigens on its surface.

The OMV of the invention typically comprises a PorB variant as described herein. The OMV of the invention may comprise a PorB variant as a high proportion of the total proteome in the OMV. For example, the OMV of the invention comprises a PorB variant in an amount that is ≥50% (i.e. 50% or more), ≥60%, ≥70%, ≥80%, ≥90% of the total proteome in the OMV.

The OMV of the invention comprises one or more additional bacterial antigens on its surface. The additional antigens may be MtrE, MetQ, fHbp, or fragments thereof. The additional bacterial antigens are typically from Neisseria gonorrhoeae, although antigens from other bacteria may be included, e.g. from Neisseria meningitidis.

The OMV of the invention may not comprise antigens that would contribute negatively to eliciting an effective immune response in the host cell. Such an antigen may be Neisseria gonorrhoeae PorB, which contributes to the evasion of immune suppression in the host cell. Hence, the OMV of the invention may not comprise the endogenous PorB from the Neisseria gonorrhoeae strain from which the OMV is isolated. The OMV of the invention may not comprise PorB from Neisseria gonorrhoeae strain FA1090. The OMV of the invention may not comprise Neisseria gonorrhoeae PorB. The OMV of the invention may not comprise a protein comprising the amino acid sequence of SEQ ID NO: 4. The OMV of the invention may not comprise a protein comprising the amino acid positions 36 to 48 (SEQ ID NO: 9), 81 to 95 (SEQ ID NO: 10), 113 to 146 (SEQ ID NO: 11), 169 to 178 (SEQ ID NO: 12), 201 to 226 (SEQ ID NO: 13), 254 to 259 (SEQ ID NO: 14), 289 to 303 (SEQ ID NO: 15) and 328 to 334 (SEQ ID NO: 16) of SEQ ID NO: 4. The OMV of the invention may not comprise SEQ ID NO: 9, SEQ ID NO: 10, SEQ ID NO: 11, SEQ ID NO: 12, SEQ ID NO: 13, SEQ ID NO: 14, SEQ ID NO: 15, or SEQ ID NO: 16. The OMV of the invention may not comprise a protein comprising the amino acid sequence of any one or more of SEQ ID NOs: 9, 10, 11, 12, 13, 14, 15, or 16. The OMV of the invention may not comprise a protein comprising the amino acid sequence of a protein encoded by SEQ ID NO: 1. The OMV of the invention may not comprise a protein comprising the amino acid sequence of a protein encoded by any one or more of SEQ ID NOs: 9, 10, 11, 12, 13, 14, 15, or 16. The OMV of the invention may comprise a protein comprising the amino acid sequence of SEQ ID NO: 4 wherein amino acid positions 36 to 48 (SEQ ID NO: 9), 81 to 95 (SEQ ID NO: 10), 113 to 146 (SEQ ID NO: 11), 169 to 178 (SEQ ID NO: 12), 201 to 226 (SEQ ID NO: 13), 254 to 259 (SEQ ID NO: 14), 289 to 303 (SEQ ID NO: 15) and 328 to 334 (SEQ ID NO: 16) of SEQ ID NO: 4 are replaced with SEQ ID NOs: 17, 18, 19, 20, 21, 22, 23 and 24, respectively.

Another antigen that may contribute negatively to eliciting an effective immune response in the host cell may be RpmM, which produces blocking antibodies that prevent binding of bactericidal antibodies to other surface proteins. Hence, the OMV of the invention may not comprise a RmpM protein. The OMV may not comprise a protein having ≥50% (i.e. 50% or more), ≥60%, ≥70%, ≥80%, ≥85%, >90%, ≥95%, ≥96%, ≥97%, ≥98%, >99% or 100% sequence identity to SEQ ID NO: 6. The OMV may not comprise or consist of a protein having an amino acid sequence of SEQ ID NO: 6. The OMV may not comprise a RmpM protein encoded by a nucleotide sequence having ≥50% (i.e. 50% or more), >60%, ≥70%, ≥80%, >85%, >90%, ≥95%, ≥96%, ≥97%, ≥98%, ≥99% or 100% sequence identity to SEQ ID NO: 3. The OMV may not comprise a RmpM protein from Neisseria gonorrhoeae, such as from Neisseria gonorrhoeae strain FA1090.

The OMV of the invention may not comprise a RmpM protein and comprises a PorB variant as described herein.

The OMV of the invention is substantially free from bacteria, e.g. whole bacteria, whether living or dead.

The OMV of the invention is between around 10 nm to 500 nm in diameter, such as 20 nm to 300 nm in diameter, or between around 25 nm to 250 nm in diameter.

The OMV of the invention is immunogenic. Hence, the OMV of the invention is capable of raising antibodies that recognise the Neisseria bacteria from which the OMV is derived, such as Neisseria gonorrhoeae, when administered to a host cell, as described further below. The OMV of the invention may be capable of eliciting an immune response, e.g. a protective immune response, such as a cell-mediated and/or an antibody response, against a pathogen (such as Neisseria bacteria), as explained further below. Preferred OMVs are those which are recognised by anti-sera upon infection with Neisseria bacteria, in particular Neisseria gonorrhoeae. More preferred are those OMVs which elicit a protective immune response against Neisseria bacteria, in particular Neisseria gonorrhoeae.

The OMV of the invention may be capable of enhancing production of pro-inflammatory cytokine IFNγ. IFNγ is the effector cytokine indicative of a Th1 response. Thus, the OMV of the invention may be capable of inducing a pro-inflammatory immune response. The OMV of the invention may be capable of inducing a Th1-biased immune response. The OMV of the invention may be capable of also priming a Th2 immune response.

The invention also provides a method of preparing an OMV, comprising purifying OMVs from the culture medium. The purification typically involves separating the OMVs from living and/or intact bacteria, for example, by size-based filtration using a filter, which allows the OMVs to pass through but which does not allow intact bacteria to pass through, or by using low speed centrifugation to pellet which allows the outer membrane vesicles in suspension. Suitable purification methods are known in the art. Particularly a two-step filtration process may be used to separate outer membrane vesicles from cell culture biomass without using centrifugation.

The method may comprise an additional step of extracting OMVs from the bacteria, such as by mechanical- or detergent-extraction, as described herein. Presence of

OMVs, as determined by size (e.g. 25 nm to 300 nm) and shape (e.g. spherical), may be determined by detection techniques known in the art, such as by microscopy or on polyacrylamide gel electrophoresis.

The invention also provides methods for preparing OMVs from more than one bacterial strain. The invention also provides a combination of OMVs from different bacterial strains. The invention also provides a kit comprising the OMV of the invention.

Components of the OMV of the invention may be determined by the components within the external surface of the OMV-producing strain, which may be modified by genetic engineering. Modified Neisseria gonorrhoeae strains useful with the invention are described herein.

### PorB Variant

The invention relates to a PorB that is a variant of the wild-type Neisseria gonorrhoeae PorB protein, i.e. a PorB variant. The OMV of the invention comprises a PorB variant. The modified Neisseria gonorrhoeae strain of the invention, from which OMVs may be obtained, expresses a PorB variant. In other words, the OMV of the invention or the modified Neisseria gonorrhoeae strain of the invention comprises a heterologous PorB protein (i.e. not the PorB protein that is endogenous to the wild-type Neisseria gonorrhoeae strain).

The PorB variant retains sufficient function to ensure viability of the modified Neisseria gonorrhoeae strain, such as its function as an outer membrane pore.

The PorB variant may comprise an amino acid sequence having 85% sequence identity to the wild-type Neisseria gonorrhoeae PorB (e.g. SEQ ID NO: 4). For example, the PorB variant may comprise an amino acid sequence having ≤50% (i.e. 50% or less), ≤60%, ≤70%, ≤80%, or ≤85% sequence identity to SEQ ID NO: 4.

The PorB variant may comprise an amino acid sequence having ≤50% (i.e. 50% or less), ≤60%, ≤70%, ≤80%, or ≤85% sequence identity to a protein encoded by the wild-type Neisseria gonorrhoeae porB gene (SEQ ID NO: 1).

The PorB variant may comprise one or more mutations compared to the wild-type Neisseria gonorrhoeae PorB protein (e.g. SEQ ID NO: 4), as explained further below.

The PorB variant may comprise or consist of a fragment of the wild-type Neisseria gonorrhoeae PorB protein. The fragment may comprise at least n consecutive amino acids from the wild-type Neisseria gonorrhoeae PorB protein and n may be 5 or more, 6 or more, 7 or more, 8 or more, 9 or more, 10 or more, 11 or more, 12 or more, 15 or more, 20 or more, 30 or more, 40 or more, 50 or more (e.g. 60, 90, 120, 150, 180, 210, 240, 270 or more). Such fragments do not comprise the full length sequence of the wild-type Neisseria gonorrhoeae PorB protein. The fragment may be 300 amino acids or less in length (for example, 290 or less, 280 or less, 270 or less, 260 or less, 250 or less, 240 or less, 230 or less, 220 or less, 210 or less, 200 or less, 190 or less, 180 or less, 170 or less, 160 or less, 150 or less, 140 or less, 130 or less, 120 or less, 110 or less, 100 or less, 50 or less, or 20 or less amino acids in length).

The PorB variant may be a PorB from another bacteria, such as a Neisseria species other than Neisseria gonorrhoeae. For example, the PorB variant may be a PorB from a Neisseria species selected from the following group: Neisseria bacilliformis, Neisseria cinerea, Neisseria elongate, Neisseria flavescens, Neisseria lactamica, Neisseria macacae, Neisseria mucosa, Neisseria oralis, Neisseria polysaccharea, Neisseria sicca, Neisseria subflava, Neisseria flava, and Neisseria meningitidis.

The PorB variant may be a PorB from Neisseria meningitidis, such as Neisseria meningitidis strain MC58.

The PorB variant may be the wild-type PorB from Neisseria meningitidis, such as from Neisseria meningitidis strain MC58 (SEQ ID NO: 5). The PorB variant may comprise or consist of an amino acid sequence having ≥50% (i.e. 50% or more), ≥60%, ≥70%, ≥80%, ≥90%, >95%, ≥98%, ≥99%, or 100% sequence identity to SEQ ID NO: 5. Where the PorB variant is a PorB from Neisseria meningitidis, the immune response may cross-react with an antigen from Neisseria meningitidis.

The PorB variant may comprise a modified Neisseria gonorrhoeae PorB (e.g. SEQ ID NO: 4), wherein the one or more extracellular loops are removed, mutated or replaced.

Where the one or more extracellular loops are replaced, they may be replaced with the extracellular loops from a PorB of another Neisseria species (e.g. Neisseria meningitides, e.g. SEQ ID NO: 5). The extracellular loops of SEQ ID NO: 4 may comprise any one or more of SEQ ID NOs: 9, 10, 11, 12, 13, 14, 15, and 16. The PorB variant may comprise an amino acid sequence which does not comprise any one or more of SEQ ID NOs: 9, 10, 11, 12, 13, 14, 15, and 16. The PorB variant may comprise an amino acid sequence of SEQ ID NO: 4 wherein the extracellular loops are replaced with the extracellular loops of SEQ ID NO: 5. The extracellular loops of SEQ ID NO: 5 may comprise any one or more of SEQ ID NOs: 17, 18, 19, 20, 21, 22, 23, or 24. The PorB variant may comprise an amino acid sequence which comprises any one or more of SEQ ID NOs: 17, 18, 19, 20, 21, 22, 23, or 24. The PorB variant may comprise an amino acid sequence of SEQ ID NO: 4 wherein the amino acid positions 36 to 48 (SEQ ID NO: 9), 81 to 95 (SEQ ID NO: 10), 113 to 146 (SEQ ID NO: 11), 169 to 178 (SEQ ID NO: 12), 201 to 226 (SEQ ID NO: 13), 254 to 259 (SEQ ID NO: 14), 289 to 303 (SEQ ID NO: 15) and 328 to 334 (SEQ ID NO: 16) of SEQ ID NO: 4 are replaced with SEQ ID NOs: 17, 18, 19, 20, 21, 22, 23 and 24, respectively.

The PorB variant may comprise one or more mutations compared to the wild-type Neisseria meningitidis PorB protein (e.g. SEQ ID NO: 5), as explained further below.

The PorB variant may be a fragment of the wild-type Neisseria meningitidis PorB. The PorB variant may be a fragment of SEQ ID NO: 5. The fragment may comprise or consist of at least n consecutive amino acids from SEQ ID NO: 5 and, n may be 5 or more, 6 or more, 7 or more, 8 or more, 9 or more, 10 or more, 11 or more, 12 or more, 15 or more, 20 or more, 30 or more, 40 or more, 50 or more (e.g. 60, 90, 120, 150, 180, 210, 240, 270 or more). Such fragments do not comprise the full length sequence of SEQ ID NO: 5. The fragment may be 300 amino acids or less in length (for example, 290 or less, 280 or less, 270 or less, 260 or less, 250 or less, 240 or less, 230 or less, 220 or less, 210 or less, 200 or less, 190 or less, 180 or less, 170 or less, 160 or less, 150 or less, 140 or less, 130 or less, 120 or less, 110 or less, 100 or less, 50 or less, or 20 or less amino acids in length).

The PorB variant may comprise or consist of an amino acid sequence having ≥50% (i.e. 50% or more), ≥60%, ≥70%, ≥80%, ≥90%, ≥95%, ≥98%, ≥99%, or 100% sequence identity to a protein encoded by the wild-type Neisseria meningitidis porB gene (SEQ ID NO: 2).

The PorB variant may comprise mutations relative to an amino acid sequence, e.g. SEQ ID NO: 4 or SEQ ID NO: 5. The mutations may each independently be a substitution, an insertion or a deletion. The PorB variant may include one or more (e.g. 1, 2, 3, 4, 5, 6, 7, 8, 9, etc.) amino acid substitutions relative to the amino acid sequence, e.g. SEQ ID NO: 4 or SEQ ID NO: 5. The PorB variant may include one or more (e.g. 1, 2, 3, 4, 5, 6, 7, 8, 9, etc.) amino acid deletions relative to the amino acid sequence, e.g. SEQ ID NO: 4 or SEQ ID NO: 5. The variant may also include one or more (e.g. 1, 2, 3, 4, 5, 6, 7, 8, 9, etc.) insertions (e.g. each of 1, 2, 3, 4 or 5 amino acids) relative to the amino acid sequence, e.g. SEQ ID NO: 4 or SEQ ID NO: 5. Deletions, substitutions or insertions may be at the N-terminus and/or C-terminus, or may be between the two termini. Thus, a truncation is an example of a deletion. Truncations may involve deletion of up to 10, up to 20, up to 30, up to 40, up to 50 (or more) amino acids at the N-terminus and/or C-terminus.

Amino acid substitutions may be to any one of the nineteen naturally occurring amino acids. In some embodiments, the one or more mutations is a conservative substitution. In another embodiment, the one or more mutations is a non-conservative substitution. A conservative substitution is commonly defined as a substitution introducing an amino acid having sufficiently similar chemical properties, e.g. having a related side chain (e.g. a basic, positively charged amino acid should be replaced by another basic, positively charged amino acid), in order to preserve the structure and the biological function of the molecule. Genetically-encoded amino acids are generally divided into four families: (1) acidic i.e. aspartate, glutamate; (2) basic i.e. lysine, arginine, histidine; (3) non-polar i.e. alanine, valine, leucine, isoleucine, proline, phenylalanine, methionine, tryptophan; and (4) uncharged polar i.e. glycine, asparagine, glutamine, cysteine, serine, threonine, tyrosine. Phenylalanine, tryptophan, and tyrosine are sometimes classified jointly as aromatic amino acids. In general, substitution of single amino acids within these families does not have a major effect on the biological activity. Further examples of conservative substitutions that may be used in the invention are presented in Table 1.

TABLE 1

| Amino Acid | Synonymous Groups | More Preferred Synonymous Groups |
|---|---|---|
| Ser | Gly, Ala, Ser, Thr, Pro | Thr, Ser |
| Arg | Asn, Lys, Gln, Arg, His | Arg, Lys, His |
| Leu | Phe, Ile, Val, Leu, Met | Ile, Val, Leu, Met |
| Pro | Gly, Ala, Ser, Thr, Pro | Pro |
| Thr | Gly, Ala, Ser, Thr, Pro | Thr, Ser |
| Ala | Gly, Thr, Pro, Ala, Ser | Gly, Ala |
| Val | Met, Phe, Ile, Leu, Val | Met, Ile, Val, Leu |
| Gly | Ala, Thr, Pro, Ser, Gly | Gly, Ala |
| Ile | Phe, Ile, Val, Leu, Met | Ile, Val, Leu, Met |
| Phe | Trp, Phe, Tyr | Tyr, Phe |
| Tyr | Trp, Phe, Tyr | Phe, Tyr |
| Cys | Ser, Thr, Cys | Cys |
| His | Asn, Lys, Gln, Arg, His | Arg, Lys, His |
| Gln | Glu, Asn, Asp, Gln | Asn, Gln |
| Asn | Glu, Asn, Asp, Gln | Asn, Gln |
| Lys | Asn, Lys, Gln, Arg, His | Arg, Lys, His |
| Asp | Glu, Asn, Asp, Gln | Asp, Glu |
| Glu | Glu, Asn, Asp, Gln | Asp, Glu |
| Met | Phe, Ile, Val, Leu, Met | Ile, Val, Leu, Met |
| Trp | Trp, Phe, Tyr | Trp |

Examples of non-conservative substitutions that may be used in the invention include the substitution of an uncharged polar amino acid with a nonpolar amino acid, the substitution of a nonpolar amino acid with an uncharged polar amino acid, the substitution of an acidic amino acid with a basic amino acid and the substitution of a basic amino acid with an acidic amino acid.

The PorB variant may comprise one or more amino acid derivatives, which includes an amino acid or amino acid-like chemical entity other than one of the 20 genetically encoded naturally occurring amino acids. In particular, the amino acid derivative may contain substituted or non-substituted, linear, branched, or cyclic alkyl moieties, and may include one or more heteroatoms. The amino acid derivatives can be made de novo or obtained from commercial sources (e.g. Calbiochem-Novabiochem AG, Switzerland; Bachem, USA).

The PorB variant may be a recombinant PorB protein. In other words, the PorB variant is expressed from recombinant DNA that is artificially introduced in the host cell. Recombinant PorB is not expressed naturally in wild-type Neisseria gonorrhoeae.

The PorB variant may be immunogenic. The immunogenicity of the PorB variant may be greater than the immunogenicity of wild-type Neisseria gonorrhoeae PorB when tested under the same conditions, such as when used in an ELISA assay or in a neutralisation assay.

Modified Neisseria gonorrhoeae Strain

The invention relates to modified Neisseria gonorrhoeae strains. Any Neisseria gonorrhoeae strain is suitable for use with the invention, such as Neisseria gonorrhoeae strain FA1090.

The modified Neisseria gonorrhoeae strain of the invention expresses a PorB variant as described herein. Hence, the modified Neisseria gonorrhoeae strain of the invention comprises in its genome a gene encoding a PorB variant as described herein.

The modified Neisseria gonorrhoeae strain of the invention may not express the endogenous porB gene, i.e. the gene encoding the PorB protein found in the Neisseria gonorrhoeae strain, such as Neisseria gonorrhoeae strain FA1090. The modified Neisseria gonorrhoeae strain of the invention does not express a protein consisting of or comprising an amino acid sequence having >50%, ≥60%, ≥70%, ≥80%, ≥85%, ≥90%, ≥91%, ≥92%, ≥93%, ≥94%, ≥95%, ≥96%, >97%, ≥98%, ≥99%, or 100% sequence identity to SEQ ID NO: 4. The modified Neisseria gonorrhoeae strain of the invention may not express a protein comprising the amino acid sequence of SEQ ID NO: 4.

The modified Neisseria gonorrhoeae strain of the invention may not comprise a nucleotide sequence having ≥50%, ≥60%, ≥70%, ≥80%, ≥85%, ≥90%, ≥91%, ≥92%, ≥93%, ≥94%, ≥95%, ≥96%, ≥97%, ≥98%, ≥99%, or 100% sequence identity to SEQ ID NO: 1. The modified Neisseria gonorrhoeae strain of the invention may not comprise a nucleotide sequence as set out in SEQ ID NO: 1. The modified Neisseria gonorrhoeae strain of the invention may not express a protein encoded by SEQ ID NO: 1.

A Neisseria gonorrhoeae strain may be modified such that it does not express the endogenous porB gene, using techniques well known in the art and as described herein.

A Neisseria gonorrhoeae strain may be modified such that the endogenous porB gene in the genome is replaced with a gene encoding the PorB variant protein described herein. Hence, the modified Neisseria gonorrhoeae strain may comprise a porB variant gene instead of the endogenous porB gene at the porB gene locus in the bacterial genome. The entire endogenous porB gene may be replaced, or portions of it may be replaced.

The modified Neisseria gonorrhoeae strain of the invention may be further modified to improve the rate at which OMVs are spontaneously released. For example, the modified Neisseria gonorrhoeae strain of the invention may not express one or more proteins involved in maintenance of membrane integrity. OMVs obtained or obtainable from such a modified strain may be referred to as generalized modules for membrane antigens (GMMA) OMVs. The OMV of the invention may be GMMA OMV.

For example, the inventors found that modified Neisseria gonorrhoeae strains which do not express RmpM generated larger amounts of OMVs. Hence, the modified Neisseria gonorrhoeae strain of the invention may not express the endogenous rmpM gene, i.e. the gene encoding the wild-type RmpM protein found in the Neisseria gonorrhoeae strain. The endogenous rmpM gene may be deleted from the genome of the modified Neisseria gonorrhoeae strain, such as Neisseria gonorrhoeae strain FA1090. The modified Neisseria gonorrhoeae strain of the invention does not express a protein consisting of or comprising an amino acid sequence having ≥50%, ≥60%, ≥70%, ≥80%, ≥85%, ≥90%, ≥91%, ≥92%, ≥93%, ≥94%, ≥95%, ≥96%, ≥97%, ≥98%, ≥99%, or 100% sequence identity to SEQ ID NO: 6. The modified Neisseria gonorrhoeae strain of the invention may not express a protein comprising the amino acid sequence as set out in SEQ ID NO: 6.

The modified Neisseria gonorrhoeae strain of the invention may not comprise a nucleotide sequence having ≥50%, ≥60%, ≥70%, ≥80%, ≥85%, ≥90%, ≥91%, ≥92%, ≥93%, ≥94%, ≥95%, ≥96%, ≥97%, ≥98%, ≥99%, or 100% sequence identity to SEQ ID NO: 3. The modified Neisseria gonorrhoeae strain of the invention may not comprise a nucleotide sequence as set out in SEQ ID NO: 3. The modified Neisseria gonorrhoeae strain of the invention may not express a protein encoded by SEQ ID NO: 3.

A Neisseria gonorrhoeae strain may be modified such that it does not express the endogenous rmpM gene. For example, the endogenous rmpM gene may be replaced with an expression cassette, as described herein. The endogenous rmpM gene may be deleted from the bacteria genome. The entire endogenous rmpM gene may be deleted, or portions of it may be deleted.

The modified Neisseria gonorrhoeae strain of the invention may not express the endogenous porB gene nor the endogenous rmpM gene. The Neisseria gonorrhoeae strain of the invention may be modified such that it does not express the endogenous porB gene nor the endogenous rmpM gene. The entire rmpM gene may be deleted from the bacteria genome and the endogenous porB gene may be replaced with a porB variant gene.

Modifications to a Neisseria gonorrhoeae bacteria strain such that it does not express an endogenous gene of interest, such as porB or rmpM, may be achieved by techniques that are within the general knowledge of the person skilled in the art. Examples of such modifications include one or more mutations in the open reading frame of the endogenous gene of interest, one or more mutations in the coding sequence of the endogenous gene of interest, one or more mutations in the endogenous promoter that controls the endogenous gene of interest gene. Modifications may include missense mutations, nonsense mutations and silent mutations. The Neisseria gonorrhoeae strain of the invention may be modified such that it does not express the endogenous gene of interest by various techniques known in the art, such as via CRISPR-mediated interference, by disrupting the endogenous gene of interest gene via gene editing, by modifying an endogenous CRISPR RNA which is designed to guide a Cas enzyme to the endogenous gene of interest, by expressing a recombinant CRISPR RNA which is designed to guide a Cas enzyme to the endogenous gene of interest.

The Neisseria gonorrhoeae strain of the invention may be modified such that the endogenous gene of interest, such as the endogenous porB or rmpM gene, is rendered non-functional. The particular endogenous gene may be rendered non-functional by mutating the gene itself or the control sequences flanking the gene, for example the promoter sequence. Deletions may remove one or more portions of the particular gene, the entire gene, and all or some of the control sequences, for example the promoter sequence. For example, deletion of only one nucleotide within the endogenous gene of interest may be made, resulting in a frame shift. However, a larger deletion may be made, for example at least about 25%, at least about 30%, at least 35%, at least 40%, at least 45%, at least about 50%, at least about 55%, at least about 60%, at least about 65%, at least about 70%, at least about 75%, at least about 80%, at least about 85%, at least about 90%, at least about 95%, or 100% of the total coding and/or non-coding sequence. Where two or more copies of the endogenous gene of interest are present in Neisseria gonorrhoeae, both copies of the gene are rendered non-functional or are deleted.

The Neisseria gonorrhoeae strain of the invention may be modified such that it is less toxic or detoxified. For example, the Neisseria gonorrhoeae strain of the invention may have a less toxic or detoxified form of LPS. For example, the Neisseria gonorrhoeae strain of the invention may have a less toxic or detoxified form of lipid A. The less toxic or detoxified Neisseria gonorrhoeae strain of the invention may be used as a vaccine, e.g. live attenuated or inactivated vaccine. Hence, the invention also provides a composition, such as an immunogenic composition (e.g. a vaccine) comprising a modified Neisseria gonorrhoeae strain of the invention e.g. a less toxic or detoxified Neisseria gonorrhoeae strain comprising mutations in its genome as described herein, and/or a fragment thereof. Such compositions, such as immunogenic compositions (e.g. vaccines) may comprise whole bacteria (e.g. live attenuated vaccine or inactivated vaccine) and/or fragment thereof, e.g. portions of the external surface of the bacteria (e.g. OMV vaccine or subunit vaccine). The invention also provides a vaccine derived from a modified Neisseria gonorrhoeae strain described herein.

The invention also provides a method of preparing the modified Neisseria gonorrhoeae strain, such as Neisseria gonorrhoeae strain FA1090, as described herein. The modified Neisseria gonorrhoeae strain of the invention is constructed using methods well known in the art. It is within the abilities of the skilled person to determine a method of preparing a bacteria strain of the invention, based on well-established molecular biology and microbiology techniques.

### Expression Cassettes

The invention also provides expression cassettes, such as expression cassettes encoding PorB variants as described herein. The invention further provides expression cassettes to remove or replace the endogenous porB or rmpM genes in the modified Neisseria gonorrhoeae strain of the invention. Any expression cassettes suitable to be expressed in bacteria can be used with the invention.

The expression cassette may comprise a gene of interest, e.g. the porB variant gene, operably linked to one or more regulatory nucleotide sequences, such as promoters which drive the expression of the downstream gene. The promoter may be any suitable promoter, such as the native promoter or an artificial promoter. The promoter may be ubiquitous, strong, weak, regulated and/or chimeric. The promoter may be a constitutive promoter. The promoter may be an inducible promoter.

For example, an expression cassette useful with the invention may comprise a native Neisseria gonorrhoeae porB promoter which is operably linked to the porB variant gene.

The expression cassette may further comprise any suitable antibiotic resistance marker for selecting transformed bacterial cells, for example kanamycin resistance marker (kanR; SEQ ID NO: 7) or erythromycin resistance marker (eryR; SEQ ID NO: 8). The antibiotic resistance marker may be driven by a promoter, e.g. a kanR promoter or an eryR promoter.

The expression cassette disclosed herein may be designed to be inserted into a locus of interest in Neisseria gonorrhoeae by homologous recombination. The locus of interest may be the endogenous porB gene locus or the endogenous rmpM gene locus. In embodiments where the expression cassette is designed to be inserted into the locus of interest by homologous recombination, the expression cassette is flanked by regions which are homologous to sequences upstream and downstream of locus of interest.

The expression cassettes disclosed herein can be prepared by amplification methods. Amplification methods include polymerase chain reaction (PCR), the ligase chain reaction (LCR), the transcription-based amplification system (TAS), the self-sustained sequence replication system (3SR). Amplified nucleotide sequences may be fused using ligation methods. In one embodiment, amplified nucleotide sequences are fused by Gibson Assembly to produce an expression cassette. A wide variety of cloning methods, host cells, and in vitro amplification methodologies are well known to the skilled person.

Transformation of Neisseria gonorrhoeae with expression cassettes can be carried out by conventional techniques. For example, transformation of Neisseria gonorrhoeae with expression cassette may be carried out using ssDNA transformation.

### Compositions

The invention also provides a composition comprising an OMV as described herein. The composition of the invention comprises OMVs obtained or obtainable from more than one modified strain of Neisseria gonorrhoeae. The composition comprises substantially, or consist essentially, of the OMVs of the invention.

The invention also provides a composition comprising a modified Neisseria gonorrhoeae strain, or a fragment thereof, as described herein.

The composition may be an immunogenic composition. The immunogenic composition is capable of eliciting antibodies when administered to a host cell. The antibodies may be capable of binding to, and neutralising, the infection and/or virulence of the Neisseria bacteria from which the composition (e.g. OMV) is derived, such as wild-type Neisseria gonorrhoeae. The immunogenic composition may be capable of eliciting an immune response, e.g. a protective immune response, such as a cell-mediated and/or an antibody response, against the Neisseria bacteria from which the composition (e.g. OMV) is derived, such as Neisseria gonorrhoeae.

The composition (e.g. immunogenic composition) of the invention may comprise an adjuvant. Any suitable adjuvant which enhances effectiveness of the composition may be used with the invention. The adjuvant may be an adsorbent. The adjuvant may be an adsorbent that does not enhance immunogenicity of OMV. The adjuvant may be an aluminium adjuvant, such as aluminium hydroxide, ALHYDROGEL®, aluminium phosphate, potassium aluminium sulphate and/or alum. A useful adjuvant for use with the invention may be oil-in-water emulsion formulations, such as for example MF59TM (containing 5% Squalene, 0.5% Tween 80, and 0.5% Span 85 formulated into submicron particles), SAF (containing 10% Squalane, 0.4% Tween 80, 5% pluronic-blocked polymer L121, and thr-MDP either microfluidized into a submicron emulsion or vortexed to generate a larger particle size emulsion), and Ribi™ adjuvant system (RAS) (containing 2% Squalene, 0.2% Tween 80, and one or more bacterial cell wall components from the group consisting of monophosphorylipid A (MPL), trehalose dimycolate (TDM), and cell wall skeleton (CW S), e.g. MPL+CWS (Detox™)). A useful adjuvant for use with the invention may be saponin adjuvants, such as Stimulon™ or particles generated therefrom such as ISCOMs (immunostimulating complexes). Other useful adjuvants include Complete Freund's Adjuvant (CFA) and Incomplete Freund's Adjuvant (IFA), cytokines, such as interleukins (eg. IL-1, IL-2, IL-4, IL-5, IL-6, IL-7, IL-12, etc.), interferons (eg. gamma interferon), macrophage colony stimulating factor (M-CSF), tumor necrosis factor (TNF), etc; and other substances that act as immunostimulating agents to enhance the effectiveness of the composition.

The composition (e.g. immunogenic composition) may be a pharmaceutical composition. The composition may further comprise a pharmaceutically acceptable excipient, carrier or diluent. The components of the pharmaceutical compositions are capable of being co-mingled with the OMVs of the present invention, and with each other, in a manner such that there is no interaction which would substantially impair the desired pharmaceutical efficacy.

Typically, the composition of the invention contains up to 85 wt % of an OMV of the invention. More typically, it contains up to 50 wt % of an OMV of the invention. Preferred pharmaceutical compositions are sterile and pyrogen free.

The composition of the invention may comprise one or more pharmaceutically acceptable salts. A “pharmaceutically acceptable salt” refers to a salt that retains the desired biological activity of the parent compound and does not impart any undesired toxicological effects. Examples of such salts include acid addition salts and base addition salts. As used herein, a pharmaceutically acceptable salt is a salt with a pharmaceutically acceptable acid or base. Pharmaceutically acceptable acids include both inorganic acids such as hydrochloric, sulphuric, phosphoric, diphosphoric, hydrobromic or nitric acid and organic acids such as oxalic, citric, fumaric, maleic, malic, ascorbic, succinic, tartaric, benzoic, acetic, methanesulphonic, ethanesulphonic, benzenesulphonic or p-toluenesulphonic acid. Pharmaceutically acceptable bases include alkali metal (e.g. sodium or potassium) and alkali earth metal (e.g. calcium or magnesium) hydroxides and organic bases such as alkyl amines, aralkyl amines and heterocyclic amines.

The composition of the invention may comprise a pharmaceutically-acceptable carrier. The pharmaceutically-acceptable carrier includes any carrier that does not itself induce the production of antibodies harmful to the individual receiving the composition Suitable carriers are typically large, slowly metabolized macromolecules such as proteins, polysaccharides, polylactic acids, polyglycolic acids, polymeric amino acids, amino acid copolymers, lipid aggregates (such as oil droplets or liposomes), and inactive virus particles. Such carriers are well known to those of ordinary skill in the art. Additionally, these carriers may function as immunostimulating agents, which may also be referred to as adjuvants.

Typically, the compositions of the invention are prepared as injectables, either as liquid solutions or suspensions, solid forms suitable for solution in, or suspension in, liquid vehicles prior to injection may also be prepared. The compositions may be lyophilized. Liquid formulations allow the compositions to be administered directly from their packaged form, without the need for reconstitution in an aqueous medium. In the embodiment where the composition is in a lyophilized form and requires reconstitution, the composition can be provided in the form of a kit which can comprise two vials, or can comprise one ready-filled syringe and one vial, with the contents of the syringe being used to reconstitute the contents of the vial prior to administration to a subject.

The composition of the invention may be provided as a kit comprising instructions to enable the kit to be used in the methods and medical uses described herein or details regarding which subjects the method may be used for.

Compositions can be presented in vials, or they can be presented in ready-filled syringes. The syringes can be supplied with or without needles. A syringe will include a single dose of the composition, whereas a vial can include a single dose or multiple doses as described herein. Kits can include a measured dose for administration to a subject.

In the embodiments where the composition of the invention comprises OMVs obtained or obtainable from more than one modified strain of Neisseria gonorrhoeae, the OMV from each modified Neisseria gonorrhoeae strain may be prepared separately prior to mixing with pharmaceutically acceptable excipients, such as buffers.

### Therapy

The OMVs and compositions of the invention are useful in therapy. Methods are disclosed herein for inducing an immune response to Neisseria gonorrhoeae in a subject using any of the disclosed compositions. The immune response can be a protective immune response. Accordingly, provided herein is an OMV or a composition as described herein, for use in medicine. The invention further relates to the use of an OMV or a composition described herein in a method for treatment of the human or animal body by therapy. The invention further relates to the use of an OMV or a composition described herein in the manufacture of a medicament.

As explained above, the OMVs and compositions provided herein the invention are useful in treating or preventing bacterial infection or a bacterial related disorder. Hence, the invention also provides a method of treating or preventing bacterial infection or a bacterial related disorder in a subject, wherein the method comprises administering to said subject an effective amount of an OMV or composition as described herein. Further provided is use of an OMV or composition as described herein for the manufacture of a medicament for use in treating or preventing bacterial infection or a bacterial related disorder.

The OMV or composition of the invention may be administered to a subject in need thereof in order to prevent the onset or reoccurrence of one or more symptoms of the bacterial infection. This is prophylaxis. Hence, the invention also provides a vaccine against Neisseria bacteria, such as Neisseria gonorrhoeae. The vaccine may have a vaccine efficacy against Neisseria gonorrhoeae of at least 10%, e.g. ≥20%, ≥30%, ≥40%, ≥50%, ≥60%, ≥70%, ≥80%, ≥85%, ≥90%, or more. The vaccine may also have a vaccine efficacy against Neisseria meningitidis of at least 10%, e.g. ≥20%, ≥30%, ≥40%, ≥50%, ≥60%, ≥70%, ≥80%, ≥85%, ≥90%, or more.

The OMV, composition or method of the invention may relate to inducing a pro-inflammatory immune response, inducing a Th1-biased immune response, and/or priming a Th2 immune response in the subject. Hence, the invention also provides a method of inducing a pro-inflammatory immune response, inducing a Th1-biased immune response, and/or priming a Th2 immune response in the subject.

A method of the invention may comprise comparing the immune response (e.g. Th1 response) elicited upon administering the OMV or composition of the invention and the immune response (e.g. Th1 response) elicited upon administering an OMV obtained from a corresponding Neisseria gonorrhoeae strain which does not contain a PorB variant. The immune response (e.g. Th1 response) may be determined according to standard techniques in the art, e.g. as described in the Examples, such as by determining the production of a pro-inflammatory cytokine (e.g. IFNγ). For example, a Th-1 biased response may be determined by an increase in the Th1 response relative to the Th1 response prior to administering the OMV or composition of the invention, or an increase in the Th1 response relative to the Th1 response that is elicited upon administering an OMV obtained from a corresponding Neisseria gonorrhoeae strain which does not contain a PorB variant. The corresponding Neisseria gonorrhoeae strain may contain the endogenous Neisseria gonorrhoeae PorB.

The modified Neisseria gonorrhoeae strain of the invention that has been modified such that it is less toxic or detoxified may also be useful in a vaccine against Neisseria bacteria, such as Neisseria gonorrhoeae. Such a less toxic or detoxified Neisseria gonorrhoeae strain may be used in the applications disclosed herein.

The bacterial infection is typically caused by a bacterial pathogen derived from a bacterial species for example a gram negative bacterium. The bacterium may be of the genus Neisseria, for example Neisseria gonorrhoeae (gonococcus) or Neisseria meningitidis (meningococcus). In a preferred embodiment, the bacterium is Neisseria gonorrhoeae. For example, the bacterial infection may be caused by Neisseria gonorrhoeae strain FA1090. The OMV or composition of the invention may be used to treat or prevent infections or bacterial related disorders caused by any one or a combination of the above-mentioned bacteria.

The bacterial infection or bacterial related disorder may be a microbial infection, upper and/or lower respiratory tract infections, skin and soft tissue infections and/or urinary tract infections.

The bacterial infection or bacterial related disorder may be gonorrhoea, disseminated gonococcemia, septic arthritis, gonococcal ophthalmia neonatorum, pelvic inflammatory disease, ectopic pregnancy, pelvic scarring (including infertility arising from pelvic scarring), pharyngitis, proctitis, and HIV. Such conditions may be particularly amenable to treatment when the microbial infection is caused by Neisseria gonorrhoeae.

The bacterial infection or bacterial related disorder may be meningitis, meningococcemia and septicaemia. Such conditions may be particularly amenable to treatment when the microbial infection is caused by Neisseria meningitidis.

The OMVs provided herein may be used as standalone agents. For example, they are particularly useful in standalone regimes for treating or preventing a bacterial infection or a bacterial related disorder, as described in more detail herein.

The OMV of the invention may be used in combination with other agents. For example, the OMV of the invention may be used in combination with vaccines against other Neisseria species, such as Neisseria meningitidis.

The further vaccine may be a vaccine against Neisseria meningitidis. The Neisseria meningitidis vaccine may comprise a saccharide antigen, which may be conjugated to a carrier protein. The saccharide antigen may be a capsular saccharide from serogroups A, C, W135 and/or Y. The composition may comprise the conjugates which are present in the MENVEO®, MENACTRA®, or NIMENRIX® products (all of which include conjugated capsular saccharides for each of serogroups A, C, W135 and Y). The Neisseria meningitidis vaccine may comprise a Neisseria meningitidis protein antigen, such as HmbR, NspA, NhhA, App, Omp85, TbpA, TbpB, and/or Cu,Zn-superoxide dismutase.

The further vaccine may be a vaccine against from Streptococcus pneumoniae, which may comprise a saccharide (typically conjugated), as in the PREVNAR and SYNFLORIX products.

The further vaccine may be a vaccine against hepatitis B virus, which may comprise the surface antigen HbsAg.

The further vaccine may be a vaccine against Bordetella pertussis, which may comprise pertussis holotoxin (PT) and filamentous haemagglutinin (FHA) from B. pertussis, optionally also in combination with pertactin and/or agglutinogens 2 and 3.

The further vaccine may be a vaccine against diphtheria (containing a diphtheria toxoid), a tetanus vaccine (containing a tetanus toxoid), a Haemophilus influenzae B (Hib) vaccine, a poliovirus vaccine, or a vaccine against human papillomavirus (HPV), hepatitis A virus, hepatitis B virus, human immunodeficiency virus (HIV), herpes simplex virus (HSV), Chlamydia trachomatis and/or Zika virus. The further vaccine may be CERVARIX® (against HPV types 16 and 18), GARDASIL® (against HPV types 6 and 11) or GARDASIL® (against HPV types 31, 33, 45, 52 and 58).

The immunogenic composition may be co-immunised with one or more further vaccines. The different vaccines can be administered either separately or as a combination. For separate administration, the vaccines will typically be administered at different sites e.g. one vaccine to the left upper arm, and a second vaccine to the right upper arm. Although the vaccines are administered separately, they may be administered at substantially the same time (e.g. during the same medical consultation or visit to a healthcare professional or vaccination centre), such as within 1 hour of each other. For administration for the multiple vaccines as a combination, a combination vaccine i.e. a single composition in which the different immunogens are admixed, may be used.

Administration may involve a single dose schedule, but will usually involve a multiple dose schedule, such as in a prime boost protocol. An initial dose and an additional dose can be administered within days, weeks, or months of each other. Suitable intervals between priming doses can be routinely determined e.g. between 4-16 weeks, such as one month or two months. The initial administration of the mixture can be followed by booster immunization of the same of different mixture, with at least one booster, such as two boosters. The method can include administering 1, 2, 3, 4, 5, 6, 7, 8, 9 or 10 doses.

The immunogenic composition of the invention may be administered parenterally, e.g. by injection, either subcutaneously, intramuscularly, or transdermally. Additional formulations suitable for other modes of administration include oral and pulmonary formulations, suppositories, and transdermal applications.

The immunogenic composition of the invention may be administered in a variety of dosage forms. Thus, it can be administered orally, for example as tablets, troches, lozenges, aqueous or oily suspensions, dispersible powders or granules.

The subject to be treated is a mammal, in particular a human. However, it may be non-human. Preferred non-human animals include, primates, such as marmosets or monkeys, commercially farmed animals, such as horses, cows, sheep or pigs, and pets, such as dogs, cats, mice, rats, guinea pigs, ferrets, gerbils or hamsters.

The subject can be any animal that is capable of being infected by a bacterium as described in more detail herein.

For prophylactic treatment, the subject is typically asymptomatic.

For example, the subject who is to be immunized is a human being, who may be any age e.g. 0-12 months old, 1-5 years old, 5-18 years old, 18-55 years old, or more than 55 years old. In one embodiment, the subject who is immunized is an adolescent (e.g. 12-18 years old) or an adult (18 years or older).

The subject may be an adolescent or adult who has been immunized against N. meningitidis in childhood (e.g. before 12 years of age), and who receives a booster dose of an immunogenic composition according to the invention to protect against N. gonorrhoeae.

The subject who is to be immunized may be at increased risk of infection with N. gonorrhoeae (e.g. at increased risk relative to the average risk in the general population). Such subjects may include (but are not limited to) those who are sexually active; those with multiple sexual partners (e.g. including sex workers); men who have sex with men (MSM); subjects with a partner who has tested positive for Neisseria gonorrhoeae; military personnel; neonates/infants whose mother was positive for Neisseria gonorrhoeae at birth (to protect against vertical transmission during delivery); and/or illegal drug users.

The subject who is to be immunized may be already seropositive for N. gonorrhoeae.

The subject who is to be immunized according to the invention may be co-immunized against one or more additional sexually-transmitted infections, for example infections and/or diseases caused by human papillomavirus (HPV), hepatitis A virus, hepatitis B virus, human immunodeficiency virus (HIV), herpes simplex virus (HSV), Chlamydia trachomatis and/or Zika virus. The subject may be co-immunised against N. gonorrhoeae and HPV. Such a co-immunisation strategy is particularly suitable for adolescent subjects.

A prophylactically effective amount of the agent or composition may be administered to a subject. The dose may be determined according to various parameters, especially according to the compound used; the age, weight and condition of the subject to be treated; the route of administration; and the required regimen. Again, a physician will be able to determine the required route of administration and dosage for any particular subject.

For example, the immunogenic composition may comprise any suitable amount of OMV per unit dose. Suitable amounts of the OMV may be from 0.1 to 200 μg per unit dose, particularly 10 μg, 20 μg, 25 μg, 50 μg or 100 μg. Per unit dose, aqueous immunogenic compositions of the invention may comprise a total concentration of OMV of less than 200 μg/ml, less than 100 μg/ml or less, 80 μg/ml or less, 50 μg/ml or less, 25 μg/ml or less, 20 μg/ml or less, 15 μg/ml or less, 10 μg/ml or less. Per unit dose, aqueous immunogenic compositions of the invention may comprise a total concentration of OMV of from 5 μg/ml to 200 μg/ml, from 5 μg/ml to 100 μg/ml, from 10 μg/ml to 100 μg/ml, from 10 μg/ml to 80 μg/ml, from 10 μg/ml to 50 μg/ml, 25 μg/ml to 50 μg/ml. Per unit dose, immunogenic compositions of the invention may comprise a total concentration of OMV of more than 100 μg/ml, more than 80 μg/ml, more than 50 μg/ml, more than 25 μg/ml, more than 20 μg/ml, more than 15 μg/ml or more than 10 μg/ml.

### Further Applications

The OMVs and compositions of the invention may be used as diagnostic reagents.

Hence, the invention also provides the use of a OMV or a composition according to the invention in the manufacture of a diagnostic reagent for detecting the presence of Neisseria bacteria, such as Neisseria gonorrhoeae. For example, the OMVs of the invention can be used in immunoassays to detect levels of antibodies to Neisseria proteins (such as Neisseria gonorrhoeae proteins), e.g. within biological samples, including for example, blood or serum samples. The OMVs of the invention may be used to raise antibodies against Neisseria bacteria, such as Neisseria gonorrhoeae. Hence, the invention also provides the use of an OMV according to the invention in the manufacture of antibodies raised against Neisseria bacteria (such as Neisseria gonorrhoeae). The anti-Neisseria antibodies can be used in immunoassays to detect antigen levels, e.g. within biological samples, including for example, blood or serum samples.

Appropriate protocols for the immunoassays described herein can be determined by the person skilled in the art. For example, the immunoassay may be based, for example, upon competition, direct reaction, or sandwich type assays. The OMVs of the invention or the antibodies raised from OMVs of the invention may be labelled e.g. using a probe, and the probe may be, for example, fluorescent, chemiluminescent, radioactive, or dye molecules. Appropriate assays which amplify the signals from the probe are known to the person skilled in the art; examples of which are assays which utilize biotin and avidin, and enzyme-labeled and mediated immunoassays, such as ELISA assays.

Kits suitable for immunodiagnosis are also provided, wherein the kit comprises the appropriate labeled reagents, including the OMVs and compositions of the invention, in suitable containers, along with the remaining reagents and materials (for example, suitable buffers, salt solutions, etc.) required for the conduct of the assay, as well as suitable set of assay instructions.

### Other

Unless defined otherwise, technical and scientific terms used herein have the same meaning as commonly understood by a person skilled in the art to which this invention belongs.

It is to be understood that different applications of the disclosed OMVs, methods or immunogenic compositions of the invention may be tailored to the specific needs in the art. It is also to be understood that the terminology used herein is for the purpose of describing the particular embodiments of the invention only, and is not intended to be limiting.

In general, the term “comprising” is intended to mean including but not limited to. For example, the phrase “An outer membrane vesicle comprising a PorB variant” should be interpreted to mean that the outer membrane vesicle comprises a PorB variant, but the outer membrane vesicle may comprise further components.

In some embodiments of the invention, the word “comprising” is replaced with the phrase “consisting of”. The term “consisting of” is intended to be limiting. For example, the phrase “An outer membrane vesicle consisting of a PorB variant” should be understood to mean that the outer membrane vesicle has a PorB variant and no further components.

In some embodiments of the invention, the word “comprising” is replaced with the phrase “consisting essentially of”. The term “consisting essentially of” means that specific further components can be present, namely those not materially affecting the essential characteristics of the subject matter.

As used herein, the term “gene” is intended to mean the nucleotide sequence encoding a protein, i.e. the coding sequence of the gene.

The term “about” or “around” when referring to a value refers to that value but within a reasonable degree of scientific error. Optionally, a value is “about x” or “around x” if it is within 10%, within 5%, or within 1% of x.

In addition, as used in this specification and the appended claims, the singular forms “a”, “an”, and “the” include plural references unless the content clearly dictates otherwise. Thus, for example, reference to “an OMV” includes two or more OMVs.

Furthermore, when referring to “≥x” herein, this means equal to or greater than x. When referring to “≤y” herein, this means equal to or less than y.

For the purpose of this invention, in order to determine the percent identity of two sequences (such as two polynucleotide or two polypeptide sequences), the sequences are aligned for optimal comparison purposes (e.g. gaps can be introduced in a first sequence for optimal alignment with a second sequence). The nucleotides at each position are then compared. When a position in the first sequence is occupied by the same nucleotide as the corresponding position in the second sequence, then the nucleotides are identical at that position. The percent identity between the two sequences is a function of the number of identical positions shared by the sequences (i.e., % identity=number of identical positions/total number of positions in the reference sequence×100).

Typically the sequence comparison is carried out over the length of the reference sequence. For example, if the user wished to determine whether a given (“test”) sequence is 95% identical to SEQ ID NO: 2, SEQ ID NO: 2 would be the reference sequence. To assess whether a sequence is at least 95% identical to SEQ ID NO: 2 (an example of a reference sequence), the skilled person would carry out an alignment over the length of SEQ ID NO: 2, and identify how many positions in the test sequence were identical to those of SEQ ID NO: 2. If at least 95% of the positions are identical, the test sequence is at least 95% identical to SEQ ID NO: 2. If the sequence is shorter than SEQ ID NO: 2, the gaps or missing positions should be considered to be non-identical positions.

The skilled person is aware of different computer programs that are available to determine the homology or identity between two sequences. For instance, a comparison of sequences and determination of percent identity between two sequences can be accomplished using a mathematical algorithm. In an embodiment, the percent identity between two amino acid or nucleic acid sequences is determined using the Needleman and Wunsch (1970) algorithm which has been incorporated into the GAP program in the Accelrys GCG software package (available at http://www.accelrys.com/products/gcg/), using either a Blosum 62 matrix or a PAM250 matrix, and a gap weight of 16, 14, 12, 10, 8, 6, or 4 and a length weight of 1, 2, 3, 4, 5, or 6. In another embodiment, the percent identity between two amino acid or nucleic acid sequences is determined by the Smith-Waterman homology search algorithm as implemented in the MPSRCH program (Oxford Molecular), using an affine gap search with parameters gap open penalty=12 and gap extension penalty=2.

All publications, patents and patent applications cited herein, whether supra or infra, are hereby incorporated by reference in their entirety.

The following examples illustrate the invention.

### Example 1—Materials and Methods

Generation of Neisseria gonorrhoeae Mutant Strains

N. gonorrhoeae strain FA1090 was routinely maintained on gonococcal base medium (GCB) agar plates or liquid media containing 1% Vitox, at 37° C. and 5% CO₂. Mutant strains were generated by spotting DNA fragments onto GCB plates, streaking Neisseria gonorrhoeae FA1090 over the spot, and incubating at 37° C. and 5% CO₂ for 8 hrs. Growth over the spot was then plated onto selective media (kanamycin at 80 μg/mL and erythromycin at 2 μg/mL, as appropriate) and incubated overnight at 37° C. and 5% CO₂. Mutation of colonies present on selective agar were confirmed by PCR and Sanger sequencing.

Replacement of Neisseria gonorrhoeae porB with Neisseria meningitidis porB

Genomic DNA from Neisseria meningitidis strain MC58 was used as the template to amplify Neisseria meningitidis MC58 porB gene (SEQ ID NO: 2) (primers: MC58 porB F and R, SEQ ID NOs: 27 and 28, respectively), adding downstream complementary overhangs to kanamycin resistance cassette aph (3) (referred to as kanR) (SEQ ID NO: 7) using primers kanR F and kanR R (SEQ ID NOs: 29 and 30, respectively). 799 base pairs (bp) of DNA upstream of the Neisseria gonorrhoeae FA1090 porB locus were amplified by PCR from FA1090 genomic DNA using primers FA1090 porB upstream F and R (SEQ ID NOs: 25 and 26, respectively), adding overhangs complementary to Neisseria meningitidis MC58 porB. 690 bp of DNA downstream of the Neisseria gonorrhoeae FA1090 porB locus (SEQ ID NO: 1) were amplified by PCR using primers FA1090 porB downstream F and R (SEQ ID NOs: 31 and 32, respectively), adding overhangs complementary to kanR. The four PCR products were fused by Gibson assembly (New England Biolabs), and the final construct was re-amplified and purified by gel extraction (Promega Wizard) before being used for the transformation of Neisseria gonorrhoeae FA1090. Insertion of the N. meningitidis porB gene was confirmed by PCR, Sanger sequencing, and Western blot analysis, with the resultant strain named FA1090 Nm porB.

Genetic Deletion of rmpM

Erythromycin resistance cassette ermC′ (referred to as eryR) (SEQ ID NO: 8) was amplified by PCR, with the addition of overhangs complementary to the upstream and downstream genetic regions surrounding Neisseria gonorrhoeae FA1090 rmpM (SEQ ID NO: 3) (primers eryR F and R, SEQ ID NOs: 37 and 38, respectively). 620 bp downstream and 578 bp upstream of FA1090 rmpM were amplified from FA1090 genomic DNA using primers rmpM downstream F and R (SEQ ID NOs: 33 and 34, respectively) and rmpM upstream F and R (SEQ ID NOs: 35 and 36, respectively). The three PCR products were fused by Gibson assembly (New England Biolabs), and the final construct was re-amplified and purified by gel extraction before being used for the transformation of Neisseria gonorrhoeae FA1090 and FA1090_{Nm porB}. Deletion of rmpM was confirmed by PCR, Sanger sequencing and Western blot, and the resultant strains named FA1090ΔrmpM and FA1090_{Nm porB}ΔrmpM.

### Production of OMVs

Neisseria gonorrhoeae FA1090ΔrmpM and FA1090_{MC58 PorB} ΔrmpM were streak plated onto GCB agar and grown overnight at 37° C. and 5% CO₂. Overnight growth was resuspended in liquid GCB to an optical density (OD₆₀₀) of 0.1 for FA1090ΔrmpM and 0.2 for FA1090_{MC58 PorB} ΔrmpM. Upon reaching an OD₆₀₀ of 1, the bacteria was pelleted by centrifugation at 3500 rpm for 20 minutes at 4° C. The supernatant was then passed through a 0.22 μM pore size filter, and the OMVs were obtained via ultracentrifugation of the supernatant at 45,000 rpm for 2 hrs at 4° C. OMVs were washed with PBS before ultracentrifugation as before, and stored at 4° C. Total protein content of OMV preparations was determined by standard BCA assay (Pierce).

### Protein Purification

mtrE and metQ were amplified from N. gonorrhoeae FA1090 and ligated into the plasmid pET14b, generating pET14b: mtrE and pET14b: metQ, respectively. For protein expression, E. coli B834 containing pET14b: mtrE or pET14b: metQ was grown in TB medium for 24 h at 22° C. and harvested by centrifugation at 5,000×g for 30 minutes at 4° C., prior to cell lysis using an EmulsiFlex-C5 homogeniser (Avestin, 15,000 lb/in²). The lysate was centrifuged at 20,000×g for 30 minutes at 4° C., and recombinant protein was bound to HisTrap columns (GE Healthcare), eluted with 300 mM imidazole, and further purified by size exclusion (AKTA, HiLoad 16/600 Superdex® 200 μg column; GE Healthcare). Protein concentration was estimated using a Nanodrop 2000c spectrophotometer (Thermo Scientific). Factor H binding protein (fHbp) v1.1 was expressed in E. coli B834 during growth at 22° C. for 24 hours with 1 mM IPTG (final concentration). Bacteria were harvested and resuspended in Buffer A (50 mM Na-phosphate pH 8.0, 300 mM NaCl, 30 mM imidazole) and fHbp purified by Nickel affinity chromatography (Chelating Sepharose Fast Flow; GE Healthcare). Columns were washed with Buffer A, then with 80:20 Buffer A: Buffer B (50 mM Na-phosphate pH 8.0, 300 mM NaCl, 300 mM imidazole) and proteins eluted in 40:60 Buffer A: Buffer B. Proteins were dialysed overnight at 4° C. into PBS, 1 mM DTT pH 8.0 with TEV protease prior to Nickel affinity chromatography to remove the HIS-GST-TEV. fHbp was eluted from Sepharose columns with Buffer B after washing with buffer C (50 mM Na-phosphate pH 6.0, 500 mM NaCl, 30 mM imidazole), and dialysed overnight at 4° C. into Tris pH 8.0. Proteins were filtered through a 0.22 μM pore size filter before use in immunisations.

Immunisation of Mice with OMVs

Six-week-old female Balb/c mice were immunised with 10 μg purified recombinant factor H binding protein v1.1 (fHbp, kindly gifted by Dr Antonio Ariza), with or without the addition of OMVs from Neisseria gonorrhoeae FA1090ΔrmpM or FA1090MC58 PorB ΔrmpM. Five mice per group were immunised by the intraperitoneal route in the following groups: i) PBS alone, ii) 10 μg fHbp alone, iii) 10 μg fHbp with 12.5 μg FA1090ΔrmpM OMVs and iv) 10 μg fHbp with 12.5 μg FA1090_{MC58 PorB} ΔrmpM OMVs. All components were diluted in PBS (200 μL total per mouse) and the preparations did not contain additional adjuvants. Mice had a total of three immunisations (day 1, day 21 and day 35), before blood was obtained for analysis on day 49. Blood was allowed to clot at room temperature for 30 minutes before centrifuging at 1,000×g, 4° C. for 10 minutes. Serum from individual mice was pooled for analysis. All animal experiments were carried out under protocols reviewed and approved by the Home Office, UK, under license number PPL P20CC6E82.

### Antibody Titre Determination by ELISA

Nunc Immuno Maxisorp microplates (Thermo Scientific) were coated with the target recombinant protein (fHbp v1.1, MtrE or MetQ, with the latter two proteins a kind gift from Dr Hayley Lavender) by adding 50 μL of 2.5 μg/mL protein diluted in PBS and incubating overnight at 4° C. 50 μL of PBS was added to six wells as the background controls. Plates were washed three times with 300 μL PBS+0.5% Tween20 (PBS-T) before blocking with 200 μL 4% bovine serum albumin diluted in PBS-T for 1 hour at 37° C. Plates were washed as before, before addition of pooled sera from OMV immunisation groups in threefold serial dilution series', and incubated for 1 hour at 37° C. Plates were washed as before, and anti-mouse IgG-HRP conjugated secondary antibody was added at a 1:10,000 dilution, and incubated for 1 hour at 37° C. After final washes, plates were developed by addition of TMB ELISA substrate followed by stop solution (BioTechne), and the absorbance read at 562 nm in a FLUOStar Omega plate reader (BMG LabTech). For AF488 conjugated secondary antibodies, fluorescence was measured using 485/520 nm for excitation/emission, respectively. To calculate endpoint titre, the baseline value was set at three times the average of the background controls. The endpoint titre is reported as the log₁₀ of the reciprocal of the serum dilution that was equal to the baseline value. Statistical significance was tested by two-way ANOVA with multiple comparisons in GraphPad Prism (GraphPad Software Inc. v.10.0).

### SDS-PAGE Gel Electrophoresis and Western Blotting

Whole Neisseria gonorrhoeae cell lysates (10 μL) or OMVs (5 μg) were separated on 12% SDS-polyacrylamide gels. Protein gels were either Coomassie stained or used for transferring proteins to 0.45 μM nitrocellulose membranes, using a semi-dry blot at 25 V for 30 minutes (BioRad). Membranes were blocked overnight in 5% milk in PBS-T at 4° C., before addition of primary antibody and incubation at room temperature for 2 hrs. After three washes in PBS-T, membranes were incubated with polyclonal goat anti-mouse immunoglobulins secondary antibody conjugated to HRP (Dako, P0447), diluted 1:10,000. After final washes, membranes were developed using Amersham ECL Western Blotting Analysis System (GE Healthcare) and exposed to Amersham Hyperfilm ECL (GE Healthcare). Primary antibodies were diluted in 5% milk in PBS-T at 1:1000 for anti-PorB, -MetQ and -MtrE monoclonal antibodies and 1:10,000 for anti-RmpM antibody.

### Splenocyte Re-Stimulation and Cytokine Quantification

Two weeks after the final immunisation, mouse spleens (five per group) were harvested and splenocytes were pressed through a 70 μm cell strainer, and suspended in RPMI-1640 supplemented with 10% Foetal Bovine Serum (FBS) and 50 μM 2-mercaptoethanol (complete RPMI). The cell suspension was pelleted by centrifugation at 300×g for 10 minutes, and the pellet was resuspended in 3 mL ACK lysis buffer for 5 minutes to remove red blood cells. Cells were washed with 10 mL complete RPMI, centrifuged as before, resuspended in PBS and counted using a haemocytometer. Splenocytes were diluted to 2×10⁶ cells/mL in complete RPMI and 1 mL was plated per well in a 24-well plate. Splenocytes were then stimulated with 2 μL per mL concanavalin A (500× stock, eBioscience), 5 μg Ng PorB OMVs, 5 μg Nm PorB OMVs, or left unstimulated, and incubated for 72 hours at 37° C. with 5% CO₂. Cell culture supernatants were stored at −80° C. until analysis. The release of cytokines interferon-gamma, IL-2, IL-4, IL-6, IL-10 and IL-17A were quantified by ELISA (Abcam) according to the manufacturer's instructions and diluting the supernatants as appropriate. Statistical significance was tested by two-way ANOVA with multiple comparisons in GraphPad Prism (GraphPad Software Inc. v.10.0).

### Serum Bactericidal Assay

N. gonorrhoeae was grown overnight on GCB agar and colonies were resuspended in PBS to an OD₆₀₀ of 0.07 (~6-7×10³ colony forming units (CFU)/mL) and incubated with serial dilutions of pooled heat-inactivated (56° C. for 1 hour) murine sera for 10 minutes at 37° C. with 5% CO₂. IgG-depleted human sera (Pel-freeze) was then added at a final concentration of 3% (v/v) and incubated at 37° C. with 5% CO₂ for 45 minutes. The dilutions were plated on GCB agar in triplicate as 10 μL spots and incubated for 20-24 hours at 37° C. with 5% CO₂. Survival was calculated as a percentage of the colonies present in the IgG-depleted human sera only control.

### Mass Spectrometry Analysis of OMVs

OMVs were subjected to trypsin digestion before separation and mass spectrometric (MS) analysis of the tryptic peptides was performed on an Vanquish™ Neo UHPLC coupled to Orbitrap Eclipse™ Tribrid™ Mass Spectrometer (Thermo Fisher Scientific). For LC-MS/MS analysis, 4 μL of tryptic sample was injected. Samples were further purified and concentrated on a trap column (C18 PepMap, 300 μm ID×5 mm, 5 μm, 100 Å; Thermo Fisher Scientific), then separated on a 15 cm analytical nano-LC column (PepMap C18, 75 μm ID×150 mm, 2 μm, 100 Å; Thermo Fisher Scientific) using a binary 60-minute linear gradient from 5% to 45% buffer B (80% acetonitrile (ACN), 0.1% formic acid (FA)) for 60 minutes, then to 99% buffer B over 1 minute and maintained at 99% for a further 10 minutes, at a flow rate of 300 nL/min. Solvent A is a buffer containing 2% ACN and 0.1% FA in water.

Orbitrap Eclipse™ Tribrid™ Mass Spectrometer equipped with a Thermo Easy-Spray capillary Emitter was used to analyse the separated peptides. Protein identification and label-free quantification (LFQ) were achieved using Proteome Discoverer (PD; version 2.5, Thermo Fisher Scientific). For protein identification, raw files were searched against a database containing canonical protein sequences of the N. meningitidis MC58 PorB and N. gonorrhoeae FA1090 (https://www.uniprot.org) with the Sequest HT search algorithm and using modified standard processing and consensus workflows. The false discovery rate (FDR) tolerance in the Percolator node was set to 0.01 for high confidence and 0.05 for medium confidence.

Absolute quantification of PorB in OMVs was carried out using the same method developed previously for quantifying porins in Bexsero (Whiting et al., 2020, Vaccine 38, 1431-1435). Briefly, PorB peptide standards, a native and a heavy (isotopically labelled) analogue, were synthesised by Thermo Scientific (HeavyPeptide AQUA Ultimate service, 97% purity; 99% isotope enrichment). The heavy peptides were 13C15N labelled; for SDYLGVNK (SEQ ID NO: 39), the valine residue was labelled, for GQEDLGNGLK (SEQ ID NO: 40), the penultimate leucine was labelled. Labelled peptide was added to a final concentration of 70 fmol/μL to the tryptic digests of OMVs before separation by LC/MS. Peak area ratios of tryptic-released native peptide/heavy peptide were compared with a PorB peptide standard curve (as described in, for example, Whiting et al., 2017, Journal of Proteomics & Enzymology, 6 (2)) for each peptide to provide a surrogate estimate of the molar content of PorB in the digest.

### Gonococcal Protein Microarray

Gonococcal microarray slides were custom-made by Arrayjet Limited, UK. Each microarray slide consisted of 16 identical blocks, with each mini-array containing 91 individually purified gonococcal proteins (derived from N. gonorrhoeae strain FA1090) and 17 control samples, printed in five repeats. Three sets of control samples were used for the slides, consisting of monoclonal mouse IgG, human IgG and EBNA-1 viral protein. Mouse and human IgGs were prepared at eight concentrations, with the highest concentration of 0.5 mg/mL and serially diluted with JetStar printing Buffer® (Arrayjet, UK) down to 0.0035 mg/mL. EBNA-1 viral protein at 0.05 mg/mL concentration was also included as a negative control. The slides were stored at 4° C. until use.

### Microarray Immunogenicity Probing and Data Acquisition

Slides were blocked with 3 mL of SuperG™ Blocking Buffer (Grace Biolabs, US) per slide and incubated for one hour at room temperature (RT). Mouse sera were diluted 1:300 with TBS, and 50 μL was added to each mini-array and incubated at 20° C. for one hour. After washing three times with 300 μL TBS-T (0.05% Tween-20) and once in TBS for 10 minutes, slides were incubated in the dark for 1 hour at 20° C. with 50 μL of goat anti-mouse IgG Fc (DyLight® 650) (ab97018, Abcam, UK) diluted 1:5000 in blocking agent. The same protocol was followed for determining IgG1 and IgG2a, using a goat anti-mouse IgG1 cross-adsorbed secondary antibody (Alexa Fluor™ 647) (A-21240, Invitrogen) and a goat anti-mouse IgG2a cross-adsorbed secondary antibody (Alexa Fluor™ 488) (A-21131, Invitrogen). Slides were rinsed in de-ionised water and dried by centrifugation at 200×g for 2 minutes. Slides were scanned in an InnoScan 710 (Innopsys, France) with the photomultiplier tube set to 40% for 635 nm. Image analysis and data quantification were carried out using Mapix-Microarray image acquisition and analysis software (v9.1.0, Innopsys, France). Microarray spot intensities were quantified using automatic background subtraction for each spot. The spot intensities for each protein were recorded in quintuplicate; arithmetic means were determined, and spot intensities for buffer-only controls were subtracted.

### Example 2—Genetic Replacement of porB

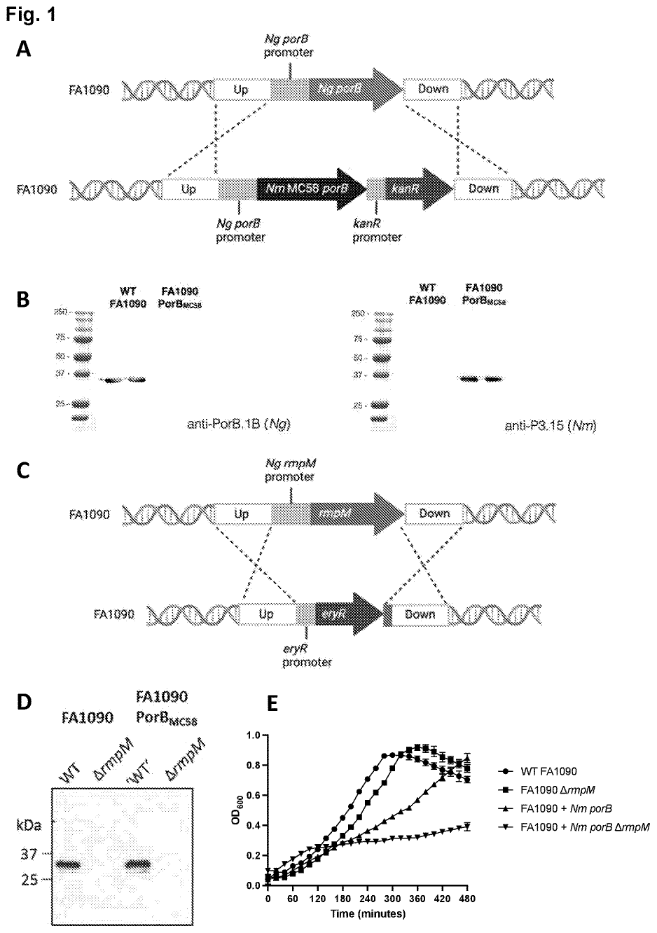

To generate Neisseria gonorrhoeae FA1090 expressing meningococcal porB, Neisseria meningitidis MC58 porB (SEQ ID NO: 2) was amplified by PCR and fused to a kanamycin resistance cassette downstream of the open reading frame. The porB-kanR construct was flanked by ~1 kb regions of sequence up- and down-stream of the porB locus in FA1090, enabling insertion by homologous recombination. The construct was introduced into FA1090 by transformation (FIG. 1A), and insertion of Nm porB was confirmed by PCR and Sanger sequencing, demonstrating that the gonococcal porB gene (SEQ ID NO: 1) had been replaced in its entirety with the meningococcal gene (SEQ ID NO: 2), with no hybrid porin generated. The resultant strain, FA1090MC58 PorB was viable, albeit with a reduced growth rate (FIG. 1E). Western blot analysis using PorB-specific typing monoclonal antibodies confirmed that FA1090MC58 PorB expressed Nm PorB alone (FIG. 1B). To address the immunomodulatory protein RmpM, the rmpM gene was removed by replacement with an erythromycin resistance cassette, and the deletion was confirmed by PCR and sequencing, as well as Western blot (FIG. 1D). The deletion of rmpM exacerbated the growth defect of FA1090MC58 PorB, although the strain remained viable (FIG. 1E).

### Example 3—OMVs from FA1090MC58 PorB Generate a Higher Antibody Titre in Mice

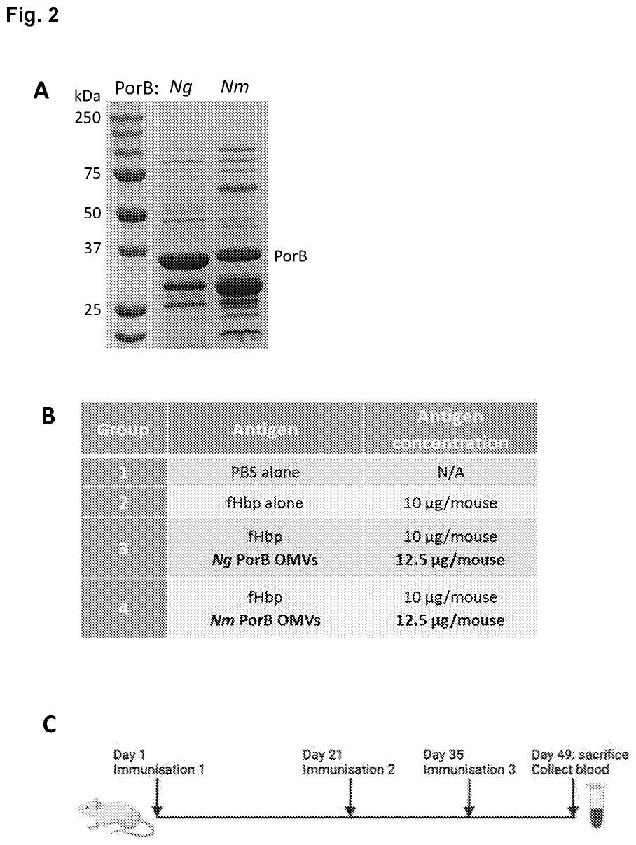

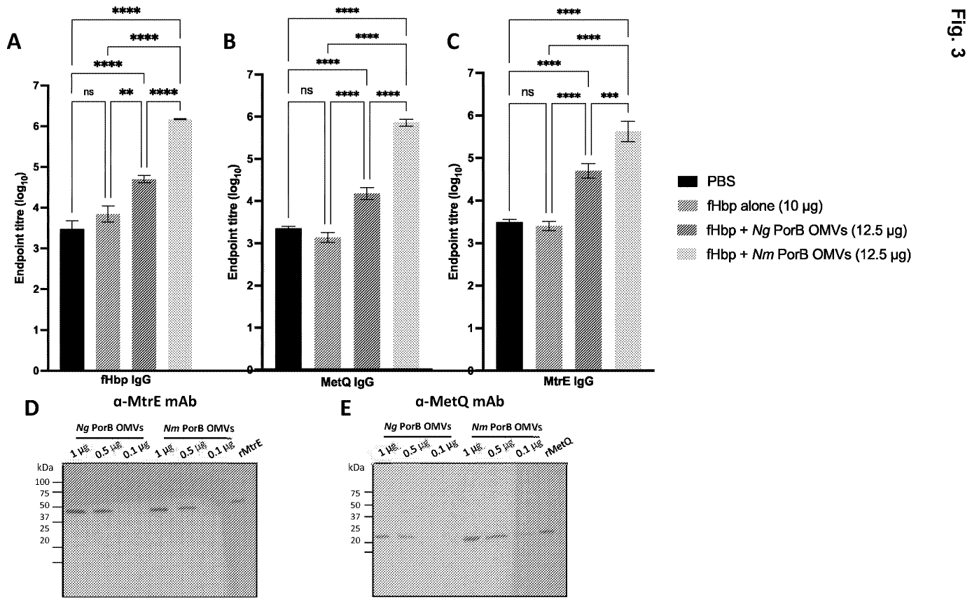

To determine the effect of replacing gonococcal PorB with meningococcal PorB on murine immune responses, OMVs were generated from FA1090ΔrmpM (‘Ng PorB OMVs’) and FA1090_{MC58 PorB} ΔrmpM (‘Nm PorB OMVs’). Balb/c mice were immunised with Ng PorB OMVs or Nm PorB OMVs in addition to a model antigen, fHbp (FIG. 2). Neisseria meningitidis fHbp does not share homology with proteins present in Neisseria gonorrhoeae OMVs, and was administered at a fixed dosage to examine the effects of the PorB replacement on murine immune responses. Anti-fHbp IgG antibody titres were significantly higher when mice were immunised with Nm PorB OMVs when compared to Ng PorB OMVs (FIG. 3A, p<0.001), suggesting that gonococcal PorB significantly contributes to immune suppression.

Antibody responses against important gonococcal OMV antigens MtrE and MetQ were also examined. MtrE is a surface exposed component of a drug efflux pump which is upregulated in AMR strains of Neisseria gonorrhoeae, while MetQ is involved in methionine uptake; both of these antigens are under evaluation for inclusion in subunit vaccines against the gonococcus. Anti-MtrE and anti-MetQ IgG antibody titres were also significantly higher when mice were immunised with Nm PorB OMVs when compared to Ng PorB OMVs (FIG. 3B-C, p<0.001). As the proteomes of Ng PorB OMVs and Nm PorB OMVs are not identical (FIG. 2A), the expression levels of MtrE and MetQ were investigated by Western blot. MtrE levels appear equal in preparations of OMVs from the different PorB expressing strains; however MetQ expression is higher in Nm PorB OMVs, and potentially influences the higher antibody titre observed (FIG. 3D-E).

Overall, these data demonstrate that Nm PorB OMVs represent a successful vaccine platform for Neisseria gonorrhoeae, by circumventing the immunomodulatory effects of gonococcal PorB so that Neisseria gonorrhoeae antigens elicit enhanced antibody responses.

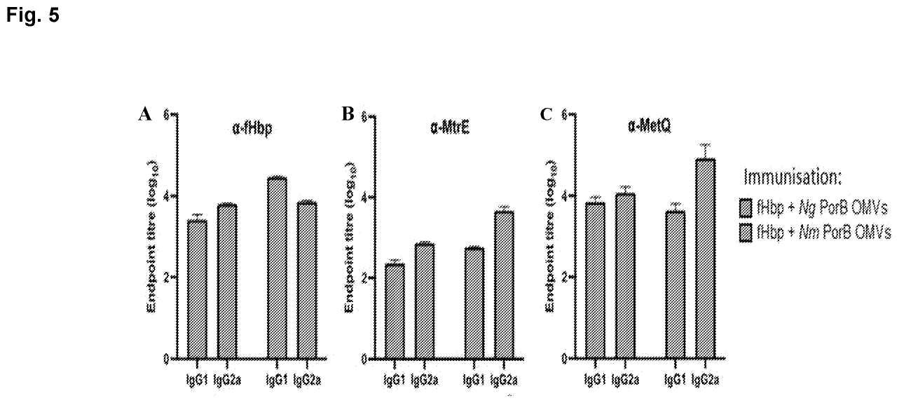

To further understand the difference in IgG responses, the ratios of IgG1 to IgG2a antibody titres were examined, as an indirect measure of Th1 vs. Th2 responses. IgG subclasses differed in their titres when comparing antibody responses to recombinant fHbp, as well as the two OMV-based antigens, MtrE and MetQ. For fHbp, IgG1 antibodies comprised a higher proportion of the total IgG titre after immunisation with Nm PorB OMVs, with an endpoint titre (log 10) of 4.5 (FIG. 5A). In contrast, for MtrE and MetQ, IgG2a antibodies represented a higher proportion of total IgG responses after immunisation with Nm PorB OMVs, with endpoint titres (log 10) of 3.7 and 4.9, respectively (FIG. 5B-C). When examining the IgG1/IgG2a ratios to indicate a Th1- or Th2-skew, immunisation with Ng PorB OMVs resulted in a Th1-bias for all three antigens examined, with IgG1/IgG2a ratios of 0.4, 0.3 and 0.6 for fHbp, MtrE and MetQ, respectively (Table 2). Immunisation with Nm PorB OMVs resulted in a Th2-bias against fHbp, with an IgG1/IgG2a ratio of 4.1, but a stronger Th1-bias against the OMV antigens MtrE and MetQ, with ratios of 0.1 and 0.05, respectively. Overall, Ng PorB OMVs elicited responses against fHbp and OMV antigens with a consistent, although small, Th1-bias. However, Nm PorB OMVs elicited a marked Th1-bias against OMV antigens, but a Th2-dominated response against the recombinant antigen, fHbp.

TABLE 2
The ratio of IgG1 to IgG2a antibody titres to
fHbp, MtrE and MetQ, elicited after
immunisation with Nm- or Ng-PorB OMVs.

| | Immunisation: | Immunisation: |
|---|---|---|
| Antigen | Ng PorB OMVs | Nm PorB OMVs |
| fHbp | 0.412 | 4.068 |
| MtrE | 0.318 | 0.121 |
| MetQ | 0.597 | 0.052 |

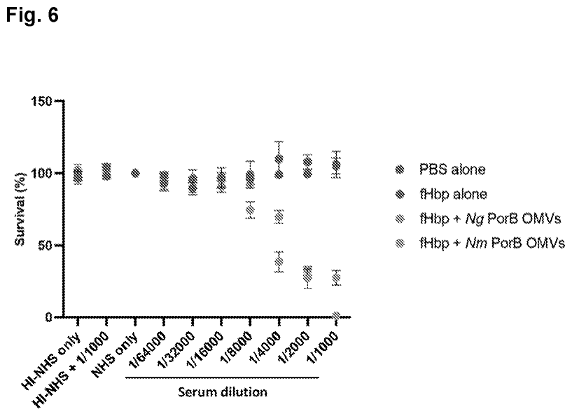

To assess the functionality of the antibody responses, serum bactericidal assays (SBA) were performed using pooled sera from OMV-immunised mice. N. gonorrhoeae FA1090 was incubated with serial dilutions of heat-inactivated sera before the addition of IgG-depleted normal human sera. Both Ng PorB OMV sera and Nm PorB OMV sera killed wild-type FA1090 in a concentration-dependent manner (FIG. 6). No bacterial killing was observed with sera from mice immunised with PBS or fHbp alone, or when using the heat-inactivated complement. SBA titres for Ng PorB OMV sera and Nm PorB OMV sera against FA1090 differed by only one dilution at 8,000 and 4,000, respectively. Overall, immunisation with Ng or Nm PorB OMVs elicited bactericidal antibodies in BALB/c mice

### Example 4—N. gonorrhoeae PorB OMVs and N. meningitidis PorB OMVs have Additional Proteome Differences

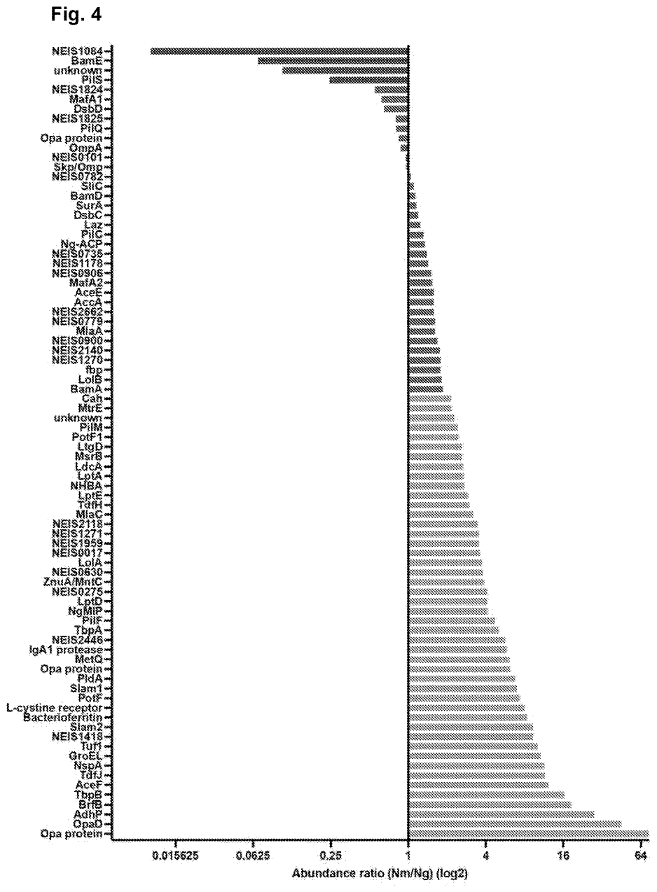

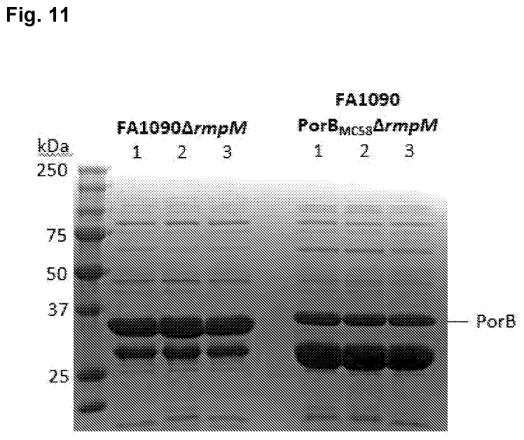

OMVs were generated from FA1090ΔrmpM (“Ng PorB OMVs”) and FA1090_{Nm porB} ΔrmpM (“Nm PorB OMVs”) as described above. Upon analysis of the OMVs by denaturing SDS-PAGE, it was observed that Ng PorB OMVs and Nm PorB OMV preparations exhibited different protein profiles, in addition to the change in PorB. Differences in the OMV proteomes were consistent between independent batches (FIG. 11). Mass spectrometry was used to compare the OMV proteomes in detail. A total of 174 proteins were identified across three biological replicates of Ng PorB OMVs and Nm PorB OMVs. Proteins identified in only one sample, with low peptide number (<2), or low overall coverage (<7%), were eliminated from the analysis, leaving 86 proteins identified with high confidence (FDR of <1%). Proteomic analysis confirmed that Ng PorB OMVs only contained FA1090 PorB, and Nm PorB OMVs only contained MC58 PorB. Other than PorB, one protein was found solely in Nm PorB OMVs, NGO_09965, an Opacity (Opa) family protein, and one protein was found solely in Ng PorB OMVs (NEIS0210, unknown function). A total of 31 out of the 86 proteins identified exhibited similar abundance in Nm PorB OMVs and Ng PorB OMVs, while 46 had a higher abundance (ratio≥2) in Nm PorB OMVs compared to Ng PorB OMVs, with only four proteins found to have lower abundance (ratio≤0.5) (FIG. 4).

Several groups of proteins with related functions exhibited a greater than two-fold increase in Nm PorB OMVs compared with Ng PorB OMVs. Iron acquisition and storage proteins transferrin binding protein B (TbpB), transferrin binding protein A (TbpA) and bacterioferritin (BrfB) were in higher abundance in Nm PorB OMVs. Furthermore, Nm PorB OMVs had a higher abundance of other nutrient acquisition proteins, including zinc-acquisition proteins TonB-dependent function protein-H and -J (TdfH, TdfJ), zinc-binding protein A (ZnuA, also known as MntC), and the methionine transporter MetQ. Potentially related to the increase in nutrient acquisition proteins, surface lipoprotein assembly modulator proteins 1 and 2 (Slam1 and Slam2) also had higher abundances; Slam1 is involved in the translocation of TbpB to the outer membrane. The abundance of metabolism-related proteins ethanol-active dehydrogenase (AdhP), dihydrolipoamide acetyltransferase (AceF), and carbonic anhydrase (Cah) were also increased in Nm PorB OMVs. A final group of interest were proteins related to host-pathogen interactions, including Neisseria surface protein A (NspA), IgAQ1 protease (NEIS1959), macrophage infectivity potentiator (Ng-MIP), and Neisseria heparin binding antigen (NHBA), which were all increased in Nm PorB OMVs compared to Ng PorB OMVs.

Two Opa-related proteins, NGO_05420 and OpaD (NEIS0903) had the largest differences when comparing Nm PorB OMVs and Ng PorB OMVs, with abundance ratios (AR, Nm/Ng) of 74 and 44, respectively. Such a large difference in AR is consistent with ON: OFF phase variation, which is known to affect Opa expression. An additional Opa54-related protein, NGO_06725, also had a higher abundance in Nm PorB OMVs, with an AR of 6.2. Additionally, NGO_07725, an Opa54-related protein, was identified with slightly lower abundance in Nm PorB OMVs with an AR of 0.84. Together these data suggest that phase variation and/or mutations occurred within the Opa coding regions during the construction of the OMV-producing strains, resulting in the expression of multiple Opa54 proteins. Overall, replacing the porB gene in N. gonorrhoeae FA1090 with meningococcal porB resulted in additional differences in the OMV proteomes with changes in factors involved in nutrient acquisition, metabolism, and host-pathogen interactions.

As a significant number of proteins were more abundant in Nm PorB OMVs, quantitative mass spectrometry was used to determine whether the abundance of PorB differed between Nm and Ng PorB OMVs. PorB represented 70.2% (±9.3%) of the Ng PorB OMV proteome, compared to 36.5% (±3.9%) of the Nm PorB OMV proteome. Quantitatively, PorB measured 506 μg/mL (±191 μg/mL) in Ng PorB OMVs and 235 μg/mL (±67 μg/mL) in Nm PorB OMVs (Table 3). In a 12.5 μg vaccine dose, PorB would average 8.8 μg (=1.2 μg) in Ng PorB OMVs and 4.6 μg (±0.5 μg) in Nm PorB OMVs. In summary, the amount of PorB is reduced in Nm PorB OMVs compared to Ng PorB OMVs, potentially reflecting the increased abundance of other proteins.

TABLE 3
Absolute quantification of PorB in Ng-
and Nm-PorB OMVs using quantitative mass
spectrometry. Data are the mean ± standard deviation.

| | PorB | Proportion | PorB in 12.5 μg |
|---|---|---|---|
| | quantification | of PorB in | immunisation |
| Sample | (μg/mL) | OMVs (%) | (μg) |
| Ng PorB OMVs | 506 ± 191 | 70.2 ± 9.3 | 8.8 ± 1.2 |
| Nm PorB OMVs | 235 ± 67 | 36.5 ± 3.9 | 4.6 ± 0.5 |

Thus, mass spectrometry demonstrated that replacing porB in N. gonorrhoeae FA1090 resulted in additional differences in the proteomes of Ng PorB OMVs and Nm PorB OMVs, other than PorB itself. The additional changes included proteins related to nutrient acquisition and host-pathogen interactions, which may be beneficial for a vaccine as these proteins are important during infection. The increased expression of nutrient acquisition proteins could relate to the growth defect of N. gonorrhoeae expressing meningococcal PorB. The proteins more highly represented in Nm PorB OMVs could contribute currently unknown immunomodulatory properties. Absolute quantification of PorB showed that the porin represented a significantly higher proportion of Ng PorB OMVs compared to Nm PorB OMVs, contributing 8.8 μg of protein compared to 4.6 μg of protein per immunisation dose, respectively. Previous data demonstrate that inhibition of T cell proliferation by gonococcal PorB is concentration dependent. Therefore, the relative reduction in PorB in Nm PorB OMVs could also contribute to the enhanced immune responses through reduced suppression of the adaptive immune responses.

### Example 5—Immunoprofiling of Murine Antibody Responses Against Gonococcal Antigens

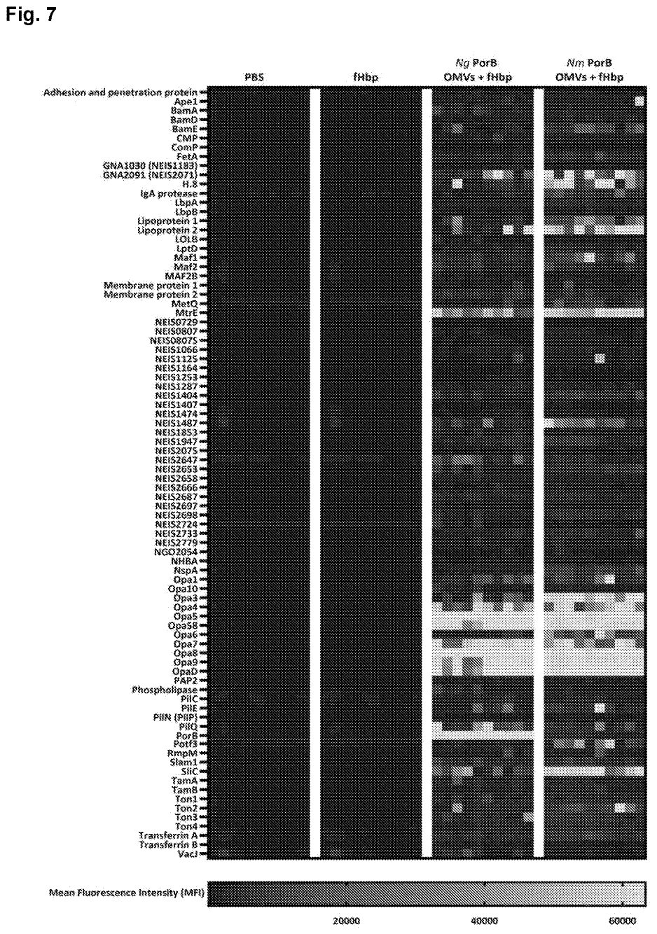

To further characterise antibody responses after immunisation with OMVs, sera from individual immunised mice were used to probe microarrays containing 91 gonococcal surface proteins. The serum IgG reactivity to each individual gonococcal antigen for mice immunised with i) PBS alone, ii) fHbp alone, iii) fHbp with Ng PorB OMVs, or iv) fHbp with Nm PorB OMVs, shown as a heat map (FIG. 7). The low background responses for the PBS and fHbp-immunised groups are readily apparent. For sera derived from mice immunised with OMVs, reactivities against multiple gonococcal antigens are detected. When comparing the Ng PorB OMV and Nm PorB OMV immunised groups, several antigens exhibit reactivities in both groups. Antigens with stronger reactivities indicated by higher mean fluorescence intensity (MFI) included MtrE, SliC, Lipoprotein 2 (NEIS0906), GNA2091 (NEIS2071), NEIS1462, Lipoprotein 1 (NEIS1063), PilQ and NEIS1487. Of these eight proteins, the antibody reactivity was stronger in the Nm PorB OMV immunised group for seven of the antigens. PilQ was the exception, where reactivity in Ng PorB OMV immunised mice was stronger. A relatively lower MFI was observed in both OMV immunised groups for BamE, PilE, and Ton2, where again the reactivity was higher in the Nm PorB OMV immunised group for three of the four antigens; for Slam1, reactivity levels were similar between Nm PorB OMV and Ng PorB OMV immunised mice. A subset of antigens showed reactivity in only one group; NEIS2647 (NGO554) displayed reactivity in only Ng PorB OMV immunised mice, whereas a larger number of antigens, including Ape1, IgA protease, Maf1, MetQ, NspA, Potf3, NEIS1125 and NEIS1405, displayed reactivity only in Nm PorB OMV immunised mice. The stronger antibody responses to MtrE and MetQ in Nm PorB OMV immunised mice, compared to Ng PorB OMV immunised mice, shown by the protein microarray are consistent with the ELISA data (FIGS. 3A-C and 5A-C). Overall, microarray data analysis demonstrated that total murine IgG antibody responses were higher and more diverse after immunisation with Nm PorB OMVs compared to immunisation with Ng PorB OMVs. The gonococcal protein microarray revealed strong reactivities to PorB and Opa variants. The microarray included PorB from FA1090 and, as expected, mice immunised with Ng PorB OMVs generated a strong antibody response to FA1090 PorB, the variant present in these OMVs. In contrast, Nm PorB OMVs elicited very weak antibody responses to FA1090 PorB. For Opa proteins, both Nm PorB OMV and Ng PorB OMV immunised mice exhibited widespread reactivity against Opa variants. The reactivity profiles to Opa variants are remarkably similar for both OMV immunised groups, particularly given the differences in Opa expression profiles revealed by proteomic analysis of the OMVs, consistent with responses elicited against epitopes shared between Opas.

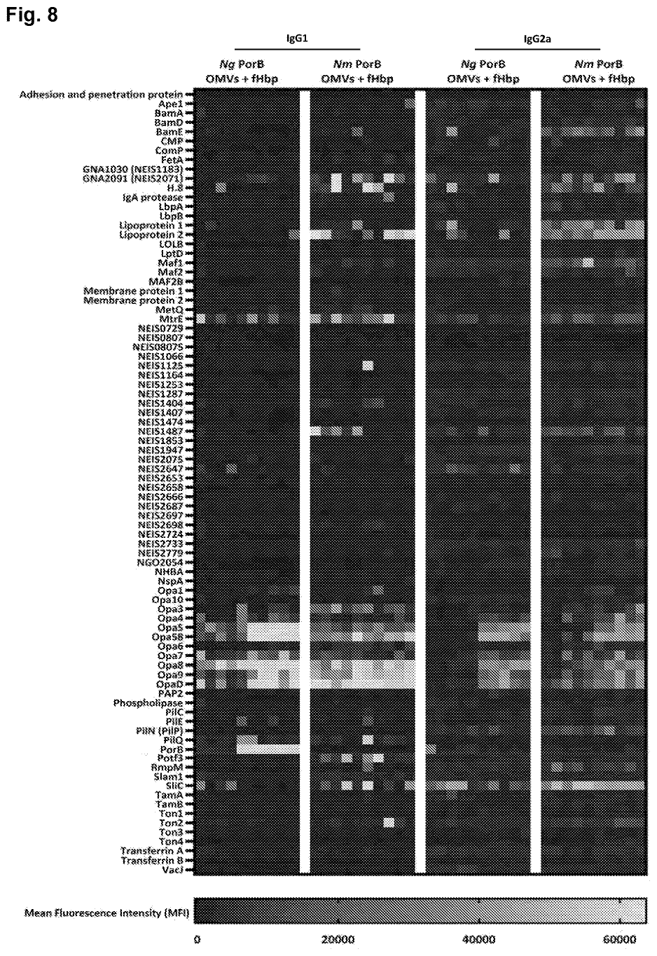

To further assess murine polyclonal IgG responses after immunisation with Ng or Nm PorB OMVs, the gonococcal protein microarrays were also used to analyse the reactivity of IgG1 and IgG2a subclasses (FIG. 8). Similar prominent profiles for IgG1 and IgG2a reactivity to the Opa variants were observed for both Ng- and Nm-PorB OMVs. Interestingly, some antigens tended to elicit IgG2a over IgG1 antibody responses; for example BamE, SliC, Lipoprotein 1 (NEIS1063) and Lipoprotein 2 (NEIS0906) have relatively stronger MFI signals for IgG2a. However, this observation was independent of the Ng/Nm PorB OMV immunisation group. Overall, when examining individual antigens, reactivity in Nm PorB OMV immunised mice was stronger for both IgG1 and IgG2a, compared to Ng PorB OMV immunised mice, suggesting that increases in both subclasses contributed to the overall higher IgG responses shown in FIG. 7.

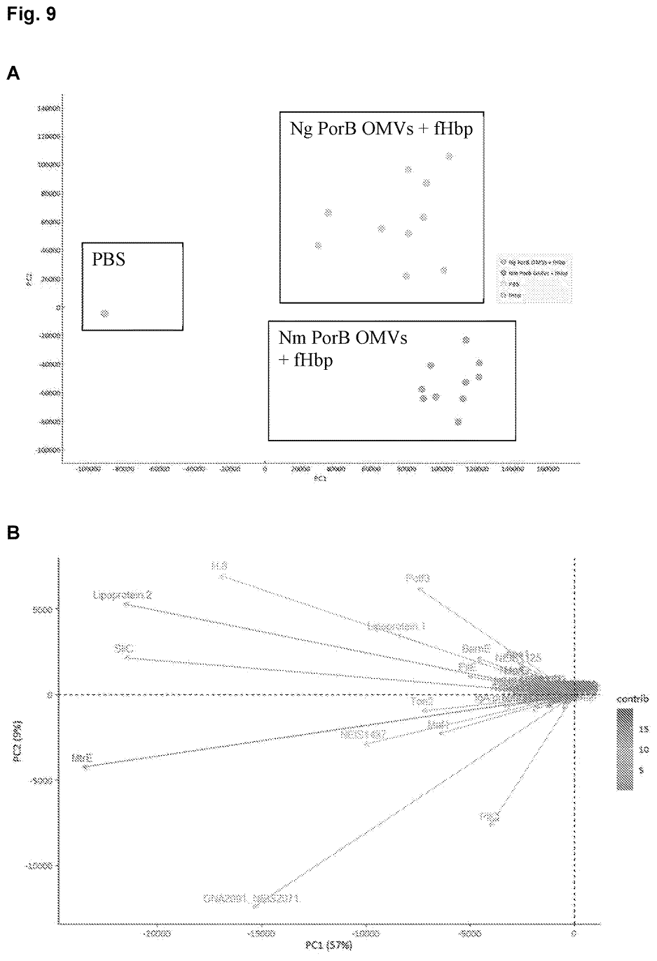

Principal Component Analysis (PCA) was applied to all four immunisation groups to capture the variance between each serum sample and grouping samples with similar reactivity profiles. As both OMV groups responded strongly to nearly all Opa proteins and only Ng PorB OMVs elicited PorB reactivity, we removed PorB and Opa variants to ascertain which other antigens were contributing to the different responses after immunisation with Ng or Nm PorB OMVs. PCA of individual serum samples showed that Ng PorB OMV and Nm PorB immunised mice were well separated from the fHbp alone and PBS control groups in the PC1 dimension (FIG. 9A; each point is a serum sample from a single mouse). Even with the PorB and Opa variants removed, sera from Nm PorB OMV immunised mice were separated from Ng PorB OMV serum samples, further from the PBS controls. This indicates a greater amplitude of antigen responses overall in the Nm PorB OMV serum samples. The antigens contributing most strongly to this separation were Lipoprotein 2 (NEIS0906), outer membrane protein H.8, Potf3, Lipoprotein 1 (NEIS1063), SliC, MtrE, GNA2091 (NEIS2071) and NEIS1487 (FIG. 9B). Stronger reactivity against SliC and BamE are particularly interesting, because SliC has a similar abundance in Ng and Nm PorB OMVs (abundance ratio of 1.1) and BamE is actually more abundant in Ng PorB OMVs (abundance ratio of 0.07). In summary, PCA showed that differences in murine IgG antibody responses to Ng PorB or Nm PorB OMVs are attributable to several different gonococcal antigens.

### Example 6—N. meningitidis PorB OMVs Elicit Pro-Inflammatory Responses in Ex Vivo Stimulated Splenocytes

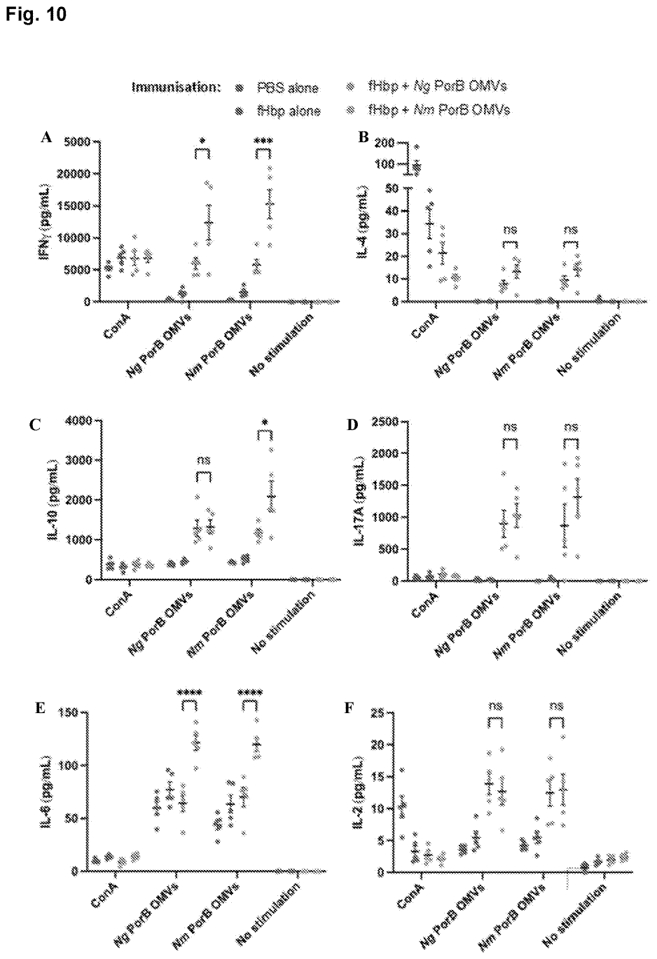

Cellular immune responses elicited by Ng/Nm PorB OMVs were investigated by re-stimulating splenocytes from immunised mice with the vaccine antigens. Production of the pro-inflammatory cytokine IFNγ, the effector cytokine indicative of a Th1 response, was significantly higher in splenocytes from Nm PorB immunised mice when compared to the Ng PorB OMV immunised group, when re-stimulated with either Ng PorB OMVs (p<0.05) or Nm PorB OMVs (p<0.001) (FIG. 10A). Overall, the increased IFNγ after immunisation with Nm PorB OMVs demonstrate that Nm PorB OMVs prime a Th1-biased response, and so further confirm the usefulness of Nm PorB OMVs in a vaccine platform for Neisseria gonorrhoeae.

IL-4 is an effector cytokine indicative of a Th2 response. There was a trend to higher IL-4 production by splenocytes from Nm PorB OMV immunised mice compared to Ng PorB OMV immunised mice when stimulated with either Ng PorB OMVs (mean 13.3 pg/mL compared to 7.7 μg/mL) or Nm PorB OMVs (mean 14.2 μg/mL compared to 9.5 μg/mL) although the differences were not statistically significant (FIG. 10B). Splenocytes from both the Ng PorB OMV and Nm PorB OMV immunised groups produced significantly more IL-4 when re-stimulated with OMVs when compared to the PBS immunised control group (p<0.0001). Together this suggests that immunisation with Ng PorB OMVs or Nm PorB OMVs primes Th2 responses to a similar extent. The production of cytokine IL-10, involved in modulating the inflammatory response, was significantly increased only in splenocytes from mice immunised with Nm PorB OMV when re-stimulated with Nm PorB OMVs (p<0.05, FIG. 10C).

Gonococcal infection drives a Th17 response, so the production of IL-17A, as an indicator of Th17 responses, was examined. Splenocytes from both the Ng PorB OMV and Nm PorB OMV immunised groups produced significantly more IL-17A when re-stimulated with OMVs when compared to the PBS immunised control group (p<0.001, FIG. 10D). However, there was no significant difference in IL-17A production between the groups of mice immunised with Ng PorB OMVs or Nm PorB OMVs (FIG. 10D), suggesting that changing PorB did not impact the murine Th17 response.

IL-6 is a pleiotropic cytokine that participates in both pro- and anti-inflammatory responses. After stimulation with Ng PorB OMVs, IL-6 production was similar in murine splenocytes from naïve mice and mice immunised with Ng PorB OMVs. However, a difference was observed between these groups when stimulated with Nm PorB OMVs, with the latter group producing more IL-6 (p<0.05). IL-6 production was also significantly higher by splenocytes from Nm PorB OMV immunised mice when compared to the Ng PorB OMV immunised group, when re-stimulated with either Ng PorB OMVs (p <0.0001) or Nm PorB OMVs (p<0.0001) (FIG. 10E). IL-2 is produced by T cells and is also a pleiotropic cytokine that does not promote any particular Th-response, shown to positively influence the differentiation, expansion, and maintenance of both Th1- and Th2-type cells, as well as T regulatory (Treg) cells and effector T cells. Production of IL-2 was significantly higher in murine splenocytes stimulated with OMVs after immunisation with either Ng PorB OMVs or Nm PorB OMVs when compared to naïve splenocytes (p<0.001), suggesting an antigen-specific expansion of T cells in response to immunisation with OMVs. However, there was no significant difference in IL-2 production between the groups of mice immunised with Ng PorB OMVs or Nm PorB OMVs (FIG. 10F).

To summarise, immunisation with Ng or Nm PorB OMVs elicits antigen-specific T cell expansion in splenocytes after re-stimulation with OMVs, evidenced by increased IL-2 production from splenocytes compared to control groups. Splenocytes from both Ng and Nm PorB OMV immunised mice elicited similar production of IL-4, suggesting a similar level of Th2 response. However, Nm PorB OMVs elicited a more pro-inflammatory response, producing significantly more IFNγ, indicative of a Th1-biased response, which is beneficial for protection against gonococcal infection. Potentially related to high IFNγ production, both regulatory cytokines IL-6 and IL-10 were significantly higher when splenocytes from Nm PorB OMV immunised mice were re-stimulated with Nm PorB OMVs.

1-2. (canceled)

3. A Neisseria gonorrhoeae OMV comprising a PorB variant.

4. The OMV of claim 3, which is obtained or obtainable from a modified Neisseria gonorrhoeae strain.

5. The OMV of claim 4, wherein the modified Neisseria gonorrhoeae strain does not express the endogenous porB gene.

6. The OMV of claim 5, wherein the endogenous porB gene is deleted from the genome of the modified Neisseria gonorrhoeae strain or wherein the endogenous porB gene in the genome of the modified Neisseria gonorrhoeae strain is replaced with a variant porB gene.

7. (canceled)

8. The OMV of claim 3, wherein the PorB variant comprises:
(a) an amino acid sequence having ≥80% sequence identity to SEQ ID NO: 5 or
(b) an amino acid sequence having ≥80% sequence identity to a protein encoded by SEQ ID NO: 2; or
(c) an amino acid sequence having ≤85% sequence identity to SEQ ID NO: 4.

9. The OMV of claim 3, wherein the PorB variant is PorB from a Neisseria species other than Neisseria gonorrhoeae, or wherein the PorB variant is from Neisseria meningitidis, or wherein the PorB variant is from Neisseria meningitidis strain MC58.

10. (canceled)

11. The OMV of claim 3, wherein the OMV does not comprise a protein having the amino acid sequence of SEQ ID NO: 4.

12. The OMV of claim 4, wherein the modified Neisseria gonorrhoeae strain does not express the endogenous rmpM gene.

13. The OMV of claim 12, wherein the endogenous rmpM gene is deleted from the genome of the modified Neisseria gonorrhoeae strain.

14. The OMV of claim 3, wherein the OMV does not comprise RmpM protein.

15. The OMV of claim 3, wherein the modified Neisseria gonorrhoeae strain is derived from FA1090.

16. The OMV of claim 3, wherein the OMV comprises MtrE, or MetQ, or MtrE and/or MetQ, or fragments thereof.

17. A modified Neisseria gonorrhoeae strain expressing a PorB variant.

18-19. (canceled)

20. An immunogenic composition comprising an OMV of claim 3, optionally wherein:
(a) the composition comprises an adjuvant, further optionally wherein the adjuvant is selected from the group consisting of: aluminium hydroxide; and/or
(b) the immunogenic composition is a pharmaceutical composition or a vaccine, optionally comprising at least one pharmaceutical carrier(s) and/or excipients.

21. (canceled)

22. A method of treating a Neisseria infection in a subject by administering a Neisseria gonorrhoeae OMV comprising a PorB variant, or the immunogenic composition comprising an OMV according to claim 20 to the subject.

23-25. (canceled)

26. The modified Neisseria gonorrhoeae strain of claim 20, wherein the PorB variant is from a Neisseria species other than Neisseria gonorrhoeae.

27. The method of claim 22, wherein the infection is a Neisseria meningitidis infection.

28. The method of claim 22, wherein the infection is a Neisseria gonorrhoeae infection.


SEQUENCE LISTING (40 sequences)

SEQ ID NO: 1 - DNA, 1047 bp, Neisseria gonorrhoeae
  source 1..1047: mol_type = genomic DNA
atgaaaaaatccctgattgccctgactttggcagcccttcctgttgcggcaatggccgatgtcaccctgtacggcgccatcaaagccggcgtacaaacttaccgttctgtagaacatacagacggcaaggtaagtaaagtggaaaccggcagcgaaatcgccgacttcggttcaaaaatcggcttcaaaggccaagaagacctcggcaacggcctgaaggccgtttggcagttggaacaaggtgcctccgtcgccggcactaacaccggctggggcaacaaacaatccttcgtcggcttgaagggcggcttcggtaccatccgcgccggtagcctgaacagccccctgaaaaacaccggcgccaacgtcaatgcttgggaatccggcaaatttaccggcaatgtgctggaaatcagcggaatggcccaacgggaacaccgctacctgtccgtacgctacgattctcccgaatttgccggcttcagcggcagcgtacaatacgcacctaaagacaattcaggctcaaacggcgaatcttaccacgttggcttgaactaccaaaacagcggcttcttcgcgcaatacgccggcttgttccaaagatacggcgaaggcactaaaaaaatcgaatacgatggtcaaacttatagtatccccagtctgtttgttgaaaaactgcaagttcaccgtttggtaggcggttacgacaataatgccctgtacgtttccgtagccgcacaacaacaagatgccaaattgtatggagcaatgagcggtaattcgcacaactctcaaaccgaagttgccgctaccgcggcataccgtttcggcaatgtaacgccccgcgtttcttacgcccacggcttcaaaggcactgttgatagtgcaaaccacgacaatacttatgaccaagtggttgtcggtgcggaatacgacttctccaaacgcacttctgccttggtttctgccggctggttgcaagaaggcaaaggcgcagacaaaatcgtatcgactgccagcgccgtcgttctgcgccacaaattctaa

SEQ ID NO: 2 - DNA, 996 bp, Neisseria meningitidis
  source 1..996: mol_type = genomic DNA
atgaaaaaatccctgattgccctgactttggcagcccttcctgttgcagcaatggctgacgttaccctgtacggcaccatcaaagccggcgtagaaacttcccgctctgtatttcaccagaacggccaagttactgaagttacaaccgctaccggcatcgttgatttgggttcgaaaatcggcttcaaaggccaagaagacctcggtaacggcctgaaagccatttggcaggttgagcaaaaagcatctatcgccggtactgactccggttggggcaaccgccaatccttcatcggcttgaaaggcggcttcggtaaattgcgcgtcggtcgtttgaacagcgtcctgaaagacaccggcgacatcaatccttgggatagcaaaagcgactatttgggtgtaaacaaaattgccgaacccgaggcacgcctcatttccgtacgctacgattctcccgaatttgccggcctcagcggcagcgtacaatacgcgcttaacgacaatgcaggcagacataacagcgaatcttaccacgccggcttcaactacaaaaacggtggcttcttcgtgcaatatggcggtgcctataaaagacatcatcaagtgcaagagggcttgaatattgagaaataccagattcaccgtttggtcagcggttacgacaatgatgccctgtacgcttccgtagccgtacagcaacaagacgcgaaactgactgatgcttccaattcgcacaactctcaaaccgaagttgccgctaccttggcataccgcttcggcaacgtaacgccccgagtttcttacgcccacggcttcaaaggtttggttgatgatgcagacataggcaacgaatacgaccaagtggttgtcggtgcggaatacgacttctccaaacgcacttctgccttggtttctgccggttggttgcaagaaggcaaaggcgaaaacaaattcgtagcgactgccggcggtgtcggtctgcgccacaaattctaa

SEQ ID NO: 3 - DNA, 711 bp, Neisseria gonorrhoeae
  source 1..711: mol_type = genomic DNA
atgaccaaacagctgaaattaagcgcattattcgttgcattgctcgcttccggcactgctgttgcgggcgaggcgtccgttcagggttacaccgtaagcggccaatcgaacgaaatcgtacgcaacaactatggagaatgctggaaaaacgcctactttgataaagcaagccaaggtcgcgtagaatgcggcgatgcggttgccgtccccgagcccgaacccgcgcctgtcgccgttgtggagcaggctcctcaatatgttgatgaaaccatttccctgtctgccaaaaccctgttcggtttcgataaggattcattgcgcgccgaagctcaagacaacctgaaagtattggcgcaacgcctgagtcgaaccaatgtccaatctgtccgcgtcgaaggccataccgactttatgggttctgaaaaatacaatcaggctctgtccgaacgccgcgcatacgtagtggcaaacaacctggtcagcaacggcgtacctgcttctagaatttctgctgtcggcttgggcgaatctcaagcgcaaatgactcaagtttgtcaagccgaagttgccaaactgggtgcgaaagcctctaaagccaaaaaacgtgaggctctgattgcatgtatcgaacctgaccgccgcgtagatgtgaaaatccgcagcatcgtaacccgtcaggttgtgccggcacgcaatcatcaccaacactaa

SEQ ID NO: 4 - protein, 348 aa, Neisseria gonorrhoeae
MKKSLIALTLAALPVAAMADVTLYGAIKAGVQTYRSVEHTDGKVSKVETGSEIADFGSKIGFKGQEDLGNGLKAVWQLEQGASVAGTNTGWGNKQSFVGLKGGFGTIRAGSLNSPLKNTGANVNAWESGKFTGNVLEISGMAQREHRYLSVRYDSPEFAGFSGSVQYAPKDNSGSNGESYHVGLNYQNSGFFAQYAGLFQRYGEGTKKIEYDGQTYSIPSLFVEKLQVHRLVGGYDNNALYVSVAAQQQDAKLYGAMSGNSHNSQTEVAATAAYRFGNVTPRVSYAHGFKGTVDSANHDNTYDQVVVGAEYDFSKRTSALVSAGWLQEGKGADKIVSTASAVVLRHKF

SEQ ID NO: 5 - protein, 331 aa, Neisseria meningitidis
MKKSLIALTLAALPVAAMADVTLYGTIKAGVETSRSVFHQNGQVTEVTTATGIVDLGSKIGFKGQEDLGNGLKAIWQVEQKASIAGTDSGWGNRQSFIGLKGGFGKLRVGRLNSVLKDTGDINPWDSKSDYLGVNKIAEPEARLISVRYDSPEFAGLSGSVQYALNDNAGRHNSESYHAGFNYKNGGFFVQYGGAYKRHHQVQEGLNIEKYQIHRLVSGYDNDALYASVAVQQQDAKLTDASNSHNSQTEVAATLAYRFGNVTPRVSYAHGFKGLVDDADIGNEYDQVVVGAEYDFSKRTSALVSAGWLQEGKGENKFVATAGGVGLRHKF

SEQ ID NO: 6 - protein, 236 aa, Neisseria gonorrhoeae
MTKQLKLSALFVALLASGTAVAGEASVQGYTVSGQSNEIVRNNYGECWKNAYFDKASQGRVECGDAVAVPEPEPAPVAVVEQAPQYVDETISLSAKTLFGFDKDSLRAEAQDNLKVLAQRLSRTNVQSVRVEGHTDFMGSEKYNQALSERRAYVVANNLVSNGVPASRISAVGLGESQAQMTQVCQAEVAKLGAKASKAKKREALIACIEPDRRVDVKIRSIVTRQVVPARNHHQH

SEQ ID NO: 7 - DNA, 966 bp, synthetic construct
  source 1..966: mol_type = other DNA
atgccgtctgaacaaccatcatcgatgaattgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggtt

SEQ ID NO: 8 - DNA, 1025 bp, synthetic construct
  source 1..1025: mol_type = other DNA
gcccttggatccgatacccccgatgacgatgcaatgagccgtctgaaatggtttcagggcaccgtgtgctctacgaccaaaagtataaaacctttaagaactttcttttttcttgtaaaaaaagaaactagataaatctctcatatcttttattcaataatcgcatcagattgcagtataaatttaacgatcactcatcatgttcatatttatcagagctcgtgctataattatactaattttataaggaggaaaaaataaagagggttataatgaacgagaaaaatataaaacacagtcaaaactttattacttcaaaacataatatagataaaataatgacaaatataagattaaatgaacatgataatatctttgaaatcggctcaggaaaagggcattttacccttgaattagtacagaggtgtaatttcgtaactgccattgaaatagaccataaattatgcaaaactacagaaaataaacttgttgatcacgataatttccaagttttaaacaaggatatattgcagtttaaatttcctaaaaaccaatcctataaaatatttggtaatataccttataacataagtacggatataatacgcaaaattgtttttgatagtatagctgatgagatttatttaatcgtggaatacgggtttgctaaaagattattaaatacaaaacgctcattggcattatttttaatggcagaagttgatatttctatattaagtatggttccaagagaatattttcatcctaaacctaaagtgaatagctcacttatcagattaaatagaaaaaaatcaagaatatcacacaaagataaacagaagtataattatttcgttatgaaatgggttaacaaagaatacaagaaaatatttacaaaaaatcaatttaacaattccttaaaacatgcaggaattgacgatttaaacaatattagctttgaacaattcttatctcttttcaatagctataaattatttaataagtaagttaagggatgcataaac

SEQ ID NO: 9 - protein, 13 aa, synthetic construct
SVEHTDGKVSKVE

SEQ ID NO: 10 - protein, 15 aa, synthetic construct
GASVAGTNTGWGNKQ

SEQ ID NO: 11 - protein, 34 aa, synthetic construct
NSPLKNTGANVNAWESGKFTGNVLEISGMAQREH

SEQ ID NO: 12 - protein, 10 aa, synthetic construct
PKDNSGSNGE

SEQ ID NO: 13 - protein, 26 aa, synthetic construct
RYGEGTKKIEYDGQTYSIPSLFVEKL

SEQ ID NO: 14 - protein, 6 aa, synthetic construct
YGAMSG

SEQ ID NO: 15 - protein, 15 aa, synthetic construct
FKGTVDSANHDNTYD

SEQ ID NO: 16 - protein, 7 aa, synthetic construct
EGKGADK

SEQ ID NO: 17 - protein, 5 aa, synthetic construct
QNGQV

SEQ ID NO: 18 - protein, 14 aa, synthetic construct
ASIAGTDSGWGNRQ

SEQ ID NO: 19 - protein, 29 aa, synthetic construct
SVLKDTGDINPWDSKSDYLGVNKIAEPEA

SEQ ID NO: 20 - protein, 10 aa, synthetic construct
NDNAGRHNSE

SEQ ID NO: 21 - protein, 13 aa, synthetic construct
RHHQVQEGLNIEK

SEQ ID NO: 22 - protein, 7 aa, synthetic construct
LTDASNS

SEQ ID NO: 23 - protein, 13 aa, synthetic construct
FKGLVDDADIGNE

SEQ ID NO: 24 - protein, 6 aa, unidentified
GKGENK

SEQ ID NO: 25 - DNA, 20 bp, synthetic construct
  source 1..20: mol_type = other DNA
cgcactgattcaagaacgca

SEQ ID NO: 26 - DNA, 37 bp, synthetic construct
  source 1..37: mol_type = other DNA
caatcagggattttttcattgctgtattccttttttg

SEQ ID NO: 27 - DNA, 37 bp, synthetic construct
  source 1..37: mol_type = other DNA
caaaaaaggaatacagcaatgaaaaaatccctgattg

SEQ ID NO: 28 - DNA, 43 bp, synthetic construct
  source 1..43: mol_type = other DNA
atgatggttgttcagacggcatttagaatttgtggcgcagacc

SEQ ID NO: 29 - DNA, 43 bp, synthetic construct
  source 1..43: mol_type = other DNA
ggtctgcgccacaaattctaaatgccgtctgaacaaccatcat

SEQ ID NO: 30 - DNA, 52 bp, synthetic construct
  source 1..52: mol_type = other DNA
gctttttgttgataccgatctttgcagaaaccaattaaccaattctgattag

SEQ ID NO: 31 - DNA, 52 bp, synthetic construct
  source 1..52: mol_type = other DNA
ctaatcagaattggttaattggtttctgcaaagatcggtatcaacaaaaagc

SEQ ID NO: 32 - DNA, 22 bp, synthetic construct
  source 1..22: mol_type = other DNA
ggggtaagttttatccacgact

SEQ ID NO: 33 - DNA, 18 bp, synthetic construct
  source 1..18: mol_type = other DNA
cacaaacggcatatcaaa

SEQ ID NO: 34 - DNA, 50 bp, synthetic construct
  source 1..50: mol_type = other DNA
atttaataagtaagttaagggatgcataaacggctaggtaatatcttgcc

SEQ ID NO: 35 - DNA, 20 bp, synthetic construct
  source 1..20: mol_type = other DNA
cgttccgcaatatccaaaac

SEQ ID NO: 36 - DNA, 47 bp, synthetic construct
  source 1..47: mol_type = other DNA
ggtatcggatccaagggctttattccctcattagatttgtacagcag

SEQ ID NO: 37 - DNA, 50 bp, synthetic construct
  source 1..50: mol_type = other DNA
ggcaagatattacctagccgtttatgcatcccttaacttacttattaaat

SEQ ID NO: 38 - DNA, 47 bp, synthetic construct
  source 1..47: mol_type = other DNA
ctgctgtacaaatctaatgagggaataaagcccttggatccgatacc

SEQ ID NO: 39 - protein, 8 aa, synthetic construct
SDYLGVNK

SEQ ID NO: 40 - protein, 10 aa, synthetic construct
GQEDLGNGLK